(19) United States
(12) Patent Application Publication
(10) Pub. No.: US 2003/0228285 A1
(43) Pub. Date: Dec. 11, 2003

(54) Bipartite T-cell factor (Tcf)-responsive promoter

(76) Inventors: Mien-Chie Hung, Houston, TX (US); Ka Yin Kwong, Rockville, MD (US); Yiyu Zou, Bronx, NY (US)

Correspondence Address: FULBRIGHT & JAWORSKI, LLP, 1301 MCKINNEY, SUITE 5100, HOUSTON, TX, 77010-3095, US

(21) Appl. No.: 10/429,802

(22) Filed: May 5, 2003

Related U.S. Application Data
(60) Provisional application No. 60/377,672, filed on May 3, 2002.

(51) Int. Cl. A61K 48/00; C07H 21/04; C12N 7/01; A61K 9/127

(52) U.S. Cl. 424/93.2; 514/44; 424/450; 435/235.1; 536/23.2

(57) ABSTRACT

The present invention is directed to methods and compositions for cancer therapy, particularly cancers resulting from a defective Wnt/β-catenin signaling pathway. In specific embodiments, a T-cell factor (Tcf)-responsive promoter regulating expression of a therapeutic gene is administered to an individual having the cancer. In a specific embodiment, the Tcf-responsive promoter comprises a minimal CMV promoter and is present on an adenovirus vector. The promoter regulates expression of a therapeutic gene.

The present invention claims priority to U.S. Provisional Patent Application 60/377,672, filed May 3, 2002, which is incorporated by reference herein in its entirety.

### FIELD OF THE INVENTION

The present invention is directed to the fields of cancer therapy and cell biology. Specifically, the present invention regards compositions and methods for cancers related to activation of the Wnt/β-catenin pathway. Specifically, the present invention regards a vector having a bipartite T-cell factor (Tcf)-responsive promoter regulating a therapeutic gene for cancer therapy.

### BACKGROUND OF THE INVENTION

Cancer is a serious health issue for millions of individuals. Colon cancer affects over 100,000 persons in the United States each year and an estimated 50,000 die of the disease during the same period (Landis et al., 1998; Landis et al., 1999). Mutation in the adenomatous polyposis coli gene (APC) or other components of the Wnt/β-catenin signaling pathway is believed to be a critical step in colon tumorigenesis. Loss of functional APC protein or constitutively stable β-catenin mutants in cancer cells prevents degradation of the β-catenin protein through the ubiqutin/proteosome pathway. As a result, β-catenin protein is accumulated in the cytoplasm and nucleus of the cancer cells, leading to hyperactivation of downstream target promoters of the Wnt/β-catenin signaling pathway (also referred to as the APC/β-catenin pathway or the β-catenin/Tcf pathway. The β-catenin protein does not bind DNA by itself; rather, it forms a bipartite complex with the T-cell factor family transcription factors and activates β-catenin/Tcf-responsive promoters. Many transcription targets of the Wnt/β-catenin signaling pathway have been identified, including genes that are involved in tumorigenesis, such as CyclinD-1 (Tetsu and McCormick, 1999; Shtutman, et al., 1999; Lin SY, et al., 2000), c-myc (He et al., 1998a), and metalloprotease (Crawford et al., 1999).

Unlike other common types of human cancers that harbor mutations in diverse pathways, mutations in the APC or β-catenin gene have been identified in most of the colon cancers (70-80%) studied so far (Goss and Groden, 2000; Polakis, 2000). On the other hand, the APC/β-catenin pathway is usually not activated in most normal tissues. Therefore, a therapeutic strategy that selectively targets this pathway is useful to most patients with primary or metastatic colon cancer.

Korinek et, al. (1997) address a stable constitutively active β-catenin-hTcf-4 complex as a result of loss of APC function, therein utilizing plasmids comprising multiple copies of a TOP sequence (a Tcf binding motif) upstream of a minimal c-Fos promoter for in vitro studies. Chen and McCormick (2001) have reported the targeting of colon cancer cells by a β-catenin/Tcf-responsive promoter in tissue culture utilizing the thymidine kinase basal promoter. The present invention addresses therapy of colon cancer in vivo and addresses an important and desirable improvement in the expression efficiency of a β-catenin/Tcf-responsive tumor-specific promoter.

### BRIEF SUMMARY OF THE INVENTION

The adenomatous polyposis coli (APC) or β-catenin genes are frequently mutated in colorectal cancers, leading to activation of downstream genes with β-catenin/T-cell factor (Tcf)-responsive promoters. The present invention addresses a gene therapy approach selectively targeting cancer cells defective in a Wnt/β-catenin pathway, such as colon cancer, colorectal cancer, or colon cancer that has metastasized to the liver. In preferred embodiments, a vector utilized for cancer therapy comprises a therapeutic gene under the control of a β-catenin/Tcf-responsive promoter. In specific embodiments, a recombinant adenovirus, such as AdTOP-CMV-TK, carries the herpes simplex virus thymidine kinase gene (HSV TK) under the control of a β-catenin/Tcf-responsive promoter. As disclosed herein, AdTOP-CMV-TK and ganciclovir (GCV) treatment significantly suppressed the growth of human DLD-1 colon cancer cells in nude mice. Furthermore, no significant tumor suppression effect was observed in an exemplary human hepatoma cell line SK-HEP-1, in which the β-catenin/Tcf pathway is not activated, indicating the therapy is selective, preferably affecting only the intended targeted cells.

In other embodiments, a T-cell factor-responsive CMV promoter-luciferase reporter (or any other reporter in the art, for example, a GFP reporter) is used to screen drugs that inhibit nuclear β-catenin activity. Other exemplary reporters include β-galactosidase, luciferase, chloramphenicol acetyltransferase, or BFP.

In some embodiments, the invention relates to nucleic acid segments comprising β-catenin/Tcf-responsive promoter construct.

The promoter construct may comprise at least two promoter regions that are operatively linked. For example, the construct may comprise a first promoter region comprising at least one Tcf/LEF-1 binding site operatively linked to a second promoter region comprising a second promoter. In some preferred embodiments, the first promoter region comprises at least three copies of a Tcf/LEF-1 binding site. Of course, the first promoter region may comprise any number of copies of Tcf/LEF-1 binding site, so long as the desired function is achieved.

In some embodiments of the invention, the second promoter region is further defined as comprising a CMV promoter, TK promoter, fos promoter, or E2F promoter. In some cases, the second promoter region will comprise a full-length promoter sequence, in other cases, the second promoter region will comprise only a minimal promoter sequence. In some preferred embodiments, the second promoter will comprise a CMV or E2F promoter. In some particularly preferred embodiments, the second promoter will comprise a minimal CMV promoter. In specific embodiments, of the invention, the β-catenin/Tcf-responsive promoter comprises at least three copies of a Tcf/LEF-1 binding site and the second promoter region comprises a minimal CMV promoter. The nucleic acid may further comprise a TOP-CMV promoter, as specifically described elsewhere in the specification.

The nucleic acid segments of the invention may further be defined as comprising a region encoding a polypeptide under the operative control of the β-catenin/Tcf-responsive promoter. For example, the polypeptide may be further defined as a therapeutic polypeptide. For example, the nucleic acid segment may comprise a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof. In cases where the region encoding a polypeptide is further defined as a suicide nucleic acid sequence, that sequence may, for example, encode thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, and/or E. coli Deo. Exemplary cancer suppressor nucleic acid sequences include p53 and/or Rb encoding sequnces. Exemplary pro-apoptotic nucleic acid sequence include p15, p16, and p21^{WAF-1} encoding sequences. Exemplary cytokine-encoding nucleic acid sequences include ones encoding granulocyte macrophage colony stimulating factor, tumor necrosis factor α, interferon α, interferon γ, IL1, IL2, IL3, IL4, IL6, IL7, IL10, IL12, and/or IL15.

The nucleic acid segment may further be defined as a vector. For example, such a vector may be a nonviral vector, a viral vector, or a combination thereof. Adenoviral vectors are preferred, in some specific embodiments. Alternative viral vectors include, but are not limited to retroviral vectors and adeno-associated vectors. Exemplary non-viral vectors include, but are not limited to, plasmids and liposomes.

Some preferred embodiments comtemplate a viral vector, comprising: a β-catenin/Tcf-responsive promoter construct comprising a first promoter region having at least one copy of a Tcf/LEF-1 binding site, operatively linked to a second promoter region; and a nucleic acid sequence encoding an amino acid sequence of interest, wherein the first and second promoter regions are operatively linked to the target nucleic acid sequence. In some particularly preferred embodiments, the viral vector is an adenoviral vector.

In some embodiments, the nucleic acid segments and/or vectors of the invention are further defined as being comprised in a pharmaceutical composition.

The invention also relates to methods of treating an individual with cancer, comprising administering to the individual a vector, said vector comprising a β-catenin/Tcf-responsive promoter construct comprising a first promoter region having at least one copy of a Tcf/LEF-1 binding site, operatively linked to a second promoter region; and a nucleic acid sequence encoding a therapeutic polypeptide, wherein the first and second promoter regions are operatively linked to the nucleic. acid sequence. With the promoter construct and therapeutic peptide being further definable as set forth above. Such methods may further comprise administering to the individual a prodrug. Exemplary prodrugs include: ganciclovir, acyclovir, FIAU [1-(2-deoxy-2-fluoro-β-D-arabinofuranosyl)-5-iodouracil], ifosfamide, 6-methoxypurine arabinoside, 5-fluorocytosine, doxorubicin, CB1954, nitrofurazone, N-(Cyanoacetyl)-L-phenylalanine, and N-(3-chloropropionyl)-L-phenylalanine.

Of course, those of skill will be able to determine a large number of cancers against which the invention may be employed. However, in some specific cases, the cancer will comprise a cell having a defective Wnt/β-catenmn pathway. In some specific embodments, the cancer is colon cancer, for example, colon cancer that has metastasized to the liver.

The methods of the invention may further comprise administering to the individual chemotherapy, radiation, surgery, or gene therapy.

In a specific embodiment, the invention relates to a method of treating colon cancer in an individual, comprising administering to the individual an adenoviral vector comprising: a β-catenin/Tcf-responsive promoter construct comprising a first promoter region having three copies of a Tcf/LEF-1 binding site, operatively linked to a minimal CMV promoter; and a nucleic acid sequence encoding thymidine kinase, wherein the first and second promoter regions are operatively linked to the nucleic acid sequence.

The invention also relates to a method of screening for a modifier of β-catenin activity, comprising providing a β-catenin/Tcf-responsive promoter construct comprising a first promoter region having at least one copy of a Tcf/LEF-1 binding site, operatively linked to a second promoter; and a reporter nucleic acid sequence, wherein the first and second promoter regions are operatively linked to the reporter nucleic acid sequence; introducing to the vector a test compound; and assaying for a change associated with the reporter nucleic acid sequence, wherein when said change occurs, said test compound is said modifier. Assaying may, in some cases, be defined as detecting transcription rate or level of said reporter nucleic acid sequence. The methods may include assaying transcription rate or level of said reporter nucleic acid sequence decreases, said test compound is an inhibitor of β-catenin activity. Exemplary reporter sequences include those encoding green fluorescent protein, blue fluorescent protein, β-galactosidase, chloramphenicol acetyltransferase, or luciferase. Exemplary test compounds include small molecules, polypeptides, polynucleotides, sugars, carbohydrates, lipids, and/or a combination thereof. The method may further be defined as occuring in a cell. The method may further comprise administering an inhibitor in a pharmaceutical composition to an individual having cancer related to a defective Wnt/β-catenin pathway.

In one embodiment of the present invention, there is a viral vector comprising a β-catenin/Tcf-responsive promoter construct comprising a first promoter region having at least one copy of a Tcf/LEF-1 binding site, operatively linked to; a second promoter region; and a nucleic acid sequence encoding an amino acid sequence of interest, wherein the first and second promoter regions are operatively linked to the target nucleic acid sequence. The vector may be further defined as an adenoviral vector. In some embodiments, the first promoter region comprises at least three copies of a Tcf/LEF-1 binding site.

In other embodiments of the present invention, the second promoter region is further defined as a minimal CMV promoter, TK promoter, fos promoter, or E2F promoter. In specific embodiments, the β-catenin/Tcf-responsive promoter comprises at least three copies of a Tcf/LEF-1 binding site and the second promoter region comprises a minimal CMV promoter. The viral vector may be further defined as comprising a TOP-CMV promoter.

In specific embodiments for any vector described herein, the nucleic acid sequence may be further defined as a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof. Exemplary suicide nucleic acid sequences include thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, or E. coli Deo, although others would be known to those of skill in the art.

In other specific embodiments for any vector described herein, a nucleic acid sequence may be further defined as encoding a cancer suppressor nucleic acid sequence, the cancer suppressor nucleic acid sequence further defined as encoding p53 or Rb.

In additional specific embodiments for any vector described herein, a nucleic acid sequence may be further defined as encoding a pro-apoptotic nucleic acid sequence, the pro-apoptotic nucleic acid sequence further defined as encoding, for example, p15, p16, or p21WAF-1, although others would be known in the art.

In other specific embodiments for any vector described herein, a nucleic acid sequence may be further defined as encoding a cytokine nucleic acid sequence, the cytokine nucleic acid sequence further defined as encoding granulocyte macrophage colony stimulating factor, tumor necrosis factor α, interferon α, interferon γ, IL1, IL2, IL3, IL4, IL6, IL7, IL10, IL12, or IL15, although others would be known in the art.

A vector, such as a viral vector, described herein may further be defined as being comprised in a pharmaceutical composition.

In other embodiments of the present invention, there is a nucleic acid segment comprising β-catenin/Tcf-responsive promoter construct comprising a first promoter region having a Tcf/LEF-1 binding site operatively linked to a second promoter, the second promoter being a minimal CMV promoter. In a specific embodiment, the first promoter region comprises at least three copies of a Tcf/LEF-1 binding site. The nucleic acid segment may be further defined as comprising a TOP-CMV promoter. In a specific embodiment, the nucleic acid segment is further defined as comprising a region encoding a polypeptide under the operative control of the β-catenin/Tcf-responsive promoter.

In a specific embodiment of the present invention, the polypeptide is further defined as a therapeutic polypeptide. A region encoding a polypeptide may be further defined as a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof. A region encoding a polypeptide may be further defined as a suicide nucleic acid sequence, exemplary embodiments of which include thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, or E. coli Deo, although others are known to those of skill in the art.

In some embodiments, a nucleic acid segment comprises a nucleic acid sequence encoding a cancer suppressor nucleic acid sequence, the cancer suppressor nucleic acid sequence further defined as encoding p53 or Rb. A nucleic acid sequence may be further defined as encoding a pro-apoptotic nucleic acid sequence, the pro-apoptotic nucleic acid sequence further defined as encoding p15, p16, or p21 WAF-1.

The nucleic acid segment may also comprise a nucleic acid sequence that encodes a cytokine nucleic acid sequence, the cytokine nucleic acid sequence further defined as encoding granulocyte macrophage colony stimulating factor, tumor necrosis factor α, interferon α, interferon γ, IL1, IL2, IL3, IL4, IL6, IL7, IL10, IL12, or IL15, although other embodiments are well known in the art.

In specific embodiments of the present invention, a nucleic acid segment is comprised in a vector, such as a nonviral vector, a viral vector, or a combination thereof. The viral vector may be an adenoviral vector, a retroviral vector, or an adeno-associated viral vector. The nonviral vector may be a plasmid or a liposome. The nucleic acid segment may also be comprised in a pharmaceutical composition.

In additional embodiments of the present invention, there is a method of treating an individual with cancer, comprising administering to the individual a vector, the vector comprising a β-catenin/Tcf-responsive promoter construct comprising a first promoter region having at least one copy of a Tcf/LEF-1 binding site, operatively linked to a second promoter region; and a nucleic acid sequence encoding a therapeutic polypeptide, wherein the first and second promoter regions are operatively linked to the nucleic acid sequence. In a specific embodiment, the first promoter region comprises at least three copies of a Tcf/LEF-1 binding site and/or the second promoter region comprises a minimal CMV promoter, TK promoter, fos promoter, or E2F promoter. In one aspect of the present invention, the β-catenin/Tcf-responsive promoter comprises at least three copies of a Tcf/LEF-1 binding site and the second promoter region comprises a minimal CMV promoter.

In a particular aspect of the present invention, the nucleic acid sequence is further defined as a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof, although other examples are known to those in the art. In a specific embodiment of the present invention, the therapeutic polypeptide is further defined as a suicide gene product.

In some embodiments, a nucleic acid sequence is further defined as encoding a suicide nucleic acid sequence, the suicide nucleic acid sequence further defined as encoding thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, or E. coli Deo.

In other specific embodiments, a nucleic acid sequence is further defined as encoding a cancer suppressor nucleic acid sequence, the cancer suppressor nucleic acid sequence further defined as encoding p53 or Rb. In one specific embodiment, the nucleic acid sequence is further defined as encoding a pro-apoptotic nucleic acid sequence, the pro-apoptotic nucleic acid sequence further defined as encoding p15, p16, or p21WAF-1. The nucleic acid sequence may be further defined as encoding a cytokine nucleic acid sequence, the cytokine nucleic acid sequence further defined as encoding granulocyte macrophage colony stimulating factor, tumor necrosis factor a, interferon a, interferon g, IL1, IL2, IL3, IL4, IL6, IL7, IL10, IL12, or IL15.

In one embodiment of the present invention, a method described herein comprises administering to an individual a prodrug, such as ganciclovir, acyclovir, FIAU [1-(2-deoxy-2-fluoro-β-D-arabinofuranosyl)-5-iodouracil], ifosfamide, 6-methoxypurine arabinoside, 5-fluorocytosine, doxorubicin, CB1954, nitrofurazone, N-(Cyanoacetyl)-L-phenylalanine, N-(3-chloropropionyl)-L-phenylalanine, or a mixture thereof, although other examples would be known in the art.

In a specific embodiment of the present invention, a cancer comprises a cell having a defective Wnt/β-catenin pathway. The cancer may be colon cancer, such as one that has metastasized to the liver. Methods of treating individuals may further comprise administering to the individual chemotherapy, radiation, surgery, or gene therapy.

In another embodiment of the present invention, there is a method of treating colon cancer in an individual, comprising administering to the individual an adenoviral vector comprising a β-catenin/Tcf-responsive promoter construct comprising a first promoter region having at least about three copies of a Tcf/LEF-1 binding site, operatively linked to a minimal CMV promoter; and a nucleic acid sequence encoding thymidine kinase, wherein the first and second promoter regions are operatively linked to the nucleic acid sequence.

In an additional embodiment of the present invention, there is a method of screening for a modifier of β-catenin activity, comprising providing a β-catenin/Tcf-responsive promoter construct comprising a first promoter region having at least one copy of a Tcf/LEF-1 binding site, operatively linked to a second promoter; and a reporter nucleic acid sequence, wherein the first and second promoter regions are operatively linked to the reporter nucleic acid sequence; introducing to the vector a test compound; and assaying for a change associated with the reporter nucleic acid sequence, wherein when the change occurs, the test compound is the modifier. In a specific embodiment, the assaying step is defined as detecting transcription rate or level of the reporter nucleic acid sequence. In a specific embodiment, the transcription rate or level of the reporter nucleic acid sequence decreases, the test compound is an inhibitor of β-catenin activity.

In a specific embodiment of the present invention, the reporter is green fluorescent protein, blue fluorescent protein, β-galactosidase, chloramphenicol acetyltransferase, or luciferase. In a specific embodiment, the second promoter is a minimal CMV promoter. In another specific embodiment, the first promoter region comprises at least three copies of a Tcf/LEF-1 binding site. The test compound may be a small molecule, a polypeptide, a polynucleotide, a sugar, a carbohydrate, a lipid, or a combination thereof, although one of skill in the art would know of other potential test compounds.

The method may be further defined as occuring in a cell and/or may further comprise administering the inhibitor in a pharmaceutical composition to an individual having cancer related to a defective Wnt/β-catenin pathway.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B illustrate β-catenin-mediated promoter activities. FIG. 1A illustrates β-catenin-activated promoters containing TOP consensus sequence in the presence of the Tcf/LEF-1 family transcription factors. FIG. 1B shows that 1.5 μg of each. plasmid was transfected into colorectal cancer cell lines DLD-1 and SW480, as well as exemplary liver cell lines Chang liver and SK-HEP-1.

FIG. 2A, FIG. 2B, and FIG. 2C demonstrate that the AdTOP-CMV-TK virus preferentially targets colon cancer cell lines in vitro. In FIG. 2A, HEK293 transfectant cell lines were infected with AdCMV-luc and AdTOP-CMV-luc viruses at various concentration (MOI, multiplicity of infection) and the luciferase activities were measured after 12 hours. In FIG. 2B, Chang Liver (not shown in this picture), SK-HEP-1, DLD-1, and SW480 cells were infected with AdTOP-CMV-TK or AdCMV-TK viruses and treated with ganciclovir (GCV) once daily for 7 days. FIG. 2C illustrates quantification of the MTT assays by measuring the absorbance at 570 nm. The data shown are the means of triplicate wells for each condition. This experiment has been repeated once and the result was consistent with data shown here.

FIG. 3A and FIG. 3B show AdTOP-CMV-TK and GCV treatment preferentially suppressed growth of β-catenin- hyperactive tumors in nude mice. In FIG. 3A, human DLD-1 colon cancer cells were infected with 25 MOI of adenoviral vectors in serum free medium. In FIG. 3B, an independent experiment was performed with human SK-HEP-1 hepatoma cells. Each mouse was inoculated with 5×10⁶ of SK-HEP-1 cells subcutaneously. Other steps were the same as in FIG. 3A.

### DETAILED DESCRIPTION OF THE INVENTION

I. DEFINITIONS

As used herein the specification, “a” or “an” may mean one or more. As used herein in the claim(s), when used in conjunction with the word “comprising”, the words “a” or “an” may mean one or more than one. As used herein “another” may mean at least a second or more.

The term “promoter” as used herein refers to a region of nucleic acid sequence that regulates expression of another nucleic acid sequence. In a specific embodiment, a promoter is a control sequence that is a region of a nucleic acid sequence at which initiation and rate of transcription are controlled. In a further specific embodiment, the promoter is bipartite, wherein two elements (promoters) concomitantly and/or in conjunction with one another, drive expression of another nucleic acid sequence located in cis on a DNA molecule.

II. THE PRESENT INVENTION

The present invention addresses a need in the art for a particularly efficient and selective system for facilitating expression of a therapeutic gene in a cancer cell having a defect in a Wnt/β-catenin pathway. More particularly, the invention regards a nucleic acid segment comprising a β-catenin/Tcf-responsive promoter, wherein this promoter comprises at least two promoter regions. In specific embodiments, the first promoter region comprises at least one copy of a Tcf/LEF-1 binding site operatively linked to a second promoter region. In one embodiment, the activity of the first promoter region comprising the Tcf/LEF-1 binding site enhances the activity of the second promoter.

A skilled artisan is aware that in embodiments wherein a specific nucleic acid or amino acid sequence is utilized, the sequence may be retrieved from publicly available databases such as the National Center for Biotechnology Information's GenBank database or from commercially available databases such as from Celera Genomics, Inc. (Rockville, Md.). In the present application, for convenience and where it is applicable, the GenBank Accession number follows the SEQ ID NOS.

In specific embodiments, the second promoter comprises the minimal cytomegalovirus (CMV) promoter (SEQ ID NO: 46; AX060694); the thymidine kinase promoter ((SEQ ID NO: 47; M15234); (SEQ ID NO: 48; M10409); SEQ ID NO: 49; M11984)), or a fragment thereof that retains promoter activity; a minimal c-Fos promoter; the c-Fos promoter (SEQ ID NO: 50; K00650); or the E2F promoter (SEQ ID NO: 51; S79170).

In a preferred embodiment, the compositions comprise a vector having a Tcf-responsive promoter. In a specific embodiment, the Tcf-responsive promoter comprises at least one Tcf/LEF-1 binding site. In a further specific embodiment, the Tcf/LEF-1 binding site is the optimal Tcf motif CCTTTGATC (SEQ ID NO: 52), as set forth in Korinek et al. (1997). In additional specific embodiments involving, for example, synthetic promoters, the reverse complement of the Tcf binding site, GATCAAAGG (SEQ ID NO: 53), will also be of use.

In a preferred embodiment, a composition of the present invention, AdTOP-CMV-TK, is utilized for the treatment of cancer.

III. β-CATENIN

β-catenin is a 92-kd protein, initially identified as a cell-cell adhesion molecule. Recent studies have indicated that β-catenin can also be translocated to the nucleus and transactivate genes whose functions are implicated in cancer formation and progression. In the past, the β-catenin pathway has been studied mainly in colon carcinoma. In 85% of colorectal cancers, the tumor suppressor adenomatous polyposis coli (“APC”) gene is lost or inactivated. Inactivation of the APC gene leads to β-catenin accumulation in the nucleus and, presumably, stimulation of tumor cell growth. Wnt signaling has now been linked to activation of the c-MYC oncogene (Pennisi, 1998). Almost 100% of colon cancers have either mutated β-catenin or deleted APC, which is expected to activate the β-catenin pathway. Barker et al. recently determined that hTcf-4 binds to β-catenin and activates transcription in colorectal epithelial cells (U.S. Pat. No. 5,998,600). Two groups identified cyclin D1 as the β-catenin target in colon carcinoma (Tetsu et al., 1999; Shtutman et al., 1999). However, it is worthwhile to mention that cyclin D1 overexpression has been found in only 30% of colon cancer ( Bartkova et al., 1994; Arber et al., 1996), which might not be consistent with almost 100% deregulation of the β-catenin pathway, suggesting that the overexpression of cyclin D1 in colon cancer may be more complicated than purely up-regulating by β-catenin.

β-catenin was first isolated as a cell-cell adhesion protein that associated with the intracellular domain of E-cadherin, a component of the adhesion junction in epithelial cells (Aberle, 1996). However, in addition to serve as an adhesion molecule, β-catenin has been shown to transduce the signals along the Wnt pathway (Fasgotto et al., 1996; Sanson et al., 1996). The transcriptional activation of target genes in response to Wnt signaling is dependent on the nuclear translocation of free cytoplasmic β-catenin and complex formation with a member of the Tcf/Lef architectural transcription factor. The regulation of this transcriptional activity is mainly achieved by strictly controlling the levels of free cytoplasmic β-catenin available for binding to the Tcf/Lef. In the absence of a Wnt signal, a quaternary cytoplasmic complex comprising β-catenin, adenomatous polyposis coli (APC), Conduction/Axin, and GSK3β mediates the phosphorylation and consequently the targeted destruction of β-catenin via the ubiquitin-proteasome pathway (Polakis, 1999). Mutation of APC in colon carcinoma or the mutations of β-catenin in a variety of cancer types could both prevent the down-regulation of β-catenin and cause constitutively activated β-catenin signaling, which contributes to the oncogenesis process effect of those cancers (Rubinfeld et al., 1997; Korinek et al., 1997; Polakis, 1999).

Mutations of APC or β-catenin in colon carcinoma cells have been found by He et al. (U.S. Pat. No. 6,140,052) and Barker (U.S. Pat. No. 5,998,600). So far, no mutation of APC or β-catenin have been found in breast cancer. However, many studies have indicated a possible role for the Wnt pathway in breast cancer. For example, mouse Wnt1, Wnt3 and Wnt10b have been found to be among the oncogenes activated by the insertion of MMTV (Musse et al., 1984; Roelink et al., 1990). Mammary hyperplasias have also occurred in Wntl transgenic mice (Tsukamoto et al., 1988). In addition, several members of the Wnt family have been shown to induce cell proliferation (Blasband et al., 1992; Wang et al., 1994). Moreover, the expression of different Wnt members has been reported to correlate with abnormal cell proliferation in human breast tissue, suggesting the possible involvement of Wnt and the β-catenin pathway in breast cancer (Dale et al., 1996; Lejeune e al., 1995; Bui et al., 1997).

In specific embodiments of the present invention, specific nucleic acid and amino acid sequences are utilized for methods and/or compositions described herein. Although a skilled artisan is aware how to retrieve such sequences from publicly available databases such as the National Center for Biotechonology Information's GenBank database, specific exemplary sequences are herein provided. Examples of β-catenin amino acid sequences, followed by their GenBank accession number, include SEQ ID NO: 1 (AAD32267); SEQ ID NO: 2 (CAA61107; CAA79497; A38973); SEQ ID NO: 3 (S35091; mouse and AAD28504; rat). Examples of β-catenin nucleic acid sequences include SEQ ID NO: 4 (X87838); SEQ ID NO: 5 (X89448); SEQ ID NO: 6 (Z19054); SEQ ID NO: 7 (AF397179) (rat); SEQ ID NO: 8 (NM-053357) (rat); and SEQ ID NO: 9 (NM_{—}007614) (mouse).

IV. CANCER TYPES

In a specific embodiment of the present invention, the methods and compositions are particularly useful in cancers having a defective Wnt/β-catenin signaling pathway. In further specific embodiments, this is defined as cancers wherein there is a mutation in APC, β-catenin, or another component of the Wnt/β-catenin signaling pathway, such as Axin1 (Satoh et al., 2000) and/or Axin2 (Liu et al,. 2000); cancers where there are constitutively stable β-catenin mutants; cancers wherein there is absence of degradation of the β-catenin protein through the ubiquitin/proteosome pathway; cancers wherein there is accumulation of β-catenin in the cytoplasm and nucleus of the cells; cancers wherein there is overexpression of β-catenin; cancers wherein there is high nuclear β-catenin activity; cancers wherein there is hyperactivation of downstream (of β-catenin) target promoters of the Wnt/β-catenin pathway; or cancers that have a combination thereof. Examples of these downstream targets of β-catenin include cyclin D1, c-myc, and metalloprotease.

In some embodiments, these cancers reside preferably in an individual having no significant activation of the Wnt/β-catenin signaling pathway in non-cancer cells. In particular embodiments, the cancers include colon cancer, colorectal cancer, colon cancer that has metastasized to the liver, breast cancer, thyroid cancer, brain cancer, head and neck cancer, prostate, liver, myelomas, bladder, blood, bone, bone-marrow, esophagus, gastrointestine, kidney, lung, nasopharynx, ovary, skin, stomach, and uterus cancers. In a preferred embodiment, the cancer is colon cancer that has metastasized to liver.

V. THERAPEUTIC GENES

The present invention is directed to providing a polynucleotide encoding a therapeutic gene product to an individual having cancer, particularly cancer related to a defective Wnt/β-catenin pathway. Chemotherapeutic suicide gene therapy approaches are known as gene-directed enzyme prodrug therapy or gene-prodrug activation therapy. Other approaches include replacement gene therapy, antisense strategies and induction of resistance to normal cells.

One skilled in the art is aware of a variety of therapeutic genes that would be beneficial for cancer therapy. In specific embodiments, therapeutic genes can include suicide genes, toxin genes, pro-apoptotic genes, cytokine genes, and/or anti-angiogenic genes. Cancer suppressor genes, including p53 and Rb, are utilized in specific embodiments. Apoptosis-inducing genes include p15, p16, and p21^{WAF-1}. Cytokine genes that may be used include GM-CSF(granulocyte macrophage colony stimulating factor), TNFα (Tumor necrosis factor α), IFN (Interferon) α, IFN γ, or IL (Interleukin) 1, IL2, IL3, IL4, IL6, IL7, IL10, IL12, or IL15 genes.

In specific methods and compositions of the present invention, the therapeutic polynucleotide is a “suicide gene” that encodes for a product causing cell death by itself or in the presence of other compounds. A representative example of such a suicide gene is one that codes for thymidine kinase of herpes simplex virus. Additional examples include thymidine kinase of varicella zoster virus, the bacterial gene cytosine deaminase (which converts 5-fluorocytosine to the highly toxic compound 5-fluorouracil), p450 oxidoreductase, carboxypeptidase G2, β-glucuronidase, penicillin-V-amidase, penicillin-G-amidase, β-lactamase, nitroreductase, carboxypeptidase A, linamarase (also referred to as β-glucosidase), the E. coli gpt gene, and the E. coli Deo gene.

In specific embodiments the suicide gene converts a prodrug into a toxic compound. As used herein, “prodrug” means any compound useful in the methods of the present invention that can be converted to a toxic product, i.e. toxic to tumor cells. The prodrug is converted to a toxic product by the gene product of the therapeutic nucleic acid sequence (suicide gene) in the vector useful in the methods of the present invention. Representative examples of such a prodrug include ganciclovir, which is converted in vivo to a toxic compound by HSV-thymidine kinase. The ganciclovir derivative subsequently is toxic to tumor cells. Other representative examples of prodrugs include ganciclovir, acyclovir, and FIAU [1-(2-deoxy-2-fluoro-β-D-arabinofuranosyl)-5-iodouracil] for thymidine kinase; ifosfamide for oxidoreductase; 6-methoxypurine arabinoside for VZV-TK; 5-fluorocytosine for cytosine deaminase; doxorubicin for β-glucuronidase; CB1954 and nitrofurazone for nitroreductase; and N-(Cyanoacetyl)-L-phenylalanine or N-(3-chloropropionyl)-L-phenylalanine for carboxypeptidase A.

The prodrug may be administered readily by a person having ordinary skill in this art. A person with ordinary skill would readily be able to determine the most appropriate dose and route for the administration of the prodrug. In specific embodiments, the prodrug is administered in a dose of from about 1-20 mg/day/kg body weight, from about 1-50 mg/day/kg body weight, or about 1-100 mg/day/kg body weight.

Exemplary nucleic acid sequences for therapeutic genes include (followed by their GenBank Accession No.): Herpes simplex virus type 1 (mutant KG111). thymidine kinase gene (SEQ ID NO: 10; J04327); Herpes simplex virus type 2 (strain 9637) thymidine kinase (tk) gene (SEQ ID NO: 11; M29941); Varicella zoster thymidine kinase (SEQ ID NO: 12; M36160); Escherichia coli cytosine deaminase (SEQ ID NO: 13; S56903); p450 oxidoreductase (SEQ ID NO: 14; D17571); carboxypeptidase G2 (SEQ ID NO: 15; M12599); β-glucuronidase (SEQ ID NO: 16; M15182); penicillin-V-amidase (SEQ ID NO: 17; M15660); penicillin-G-amidase (SEQ ID NO: 18; AF161313); β-lactamase (SEQ ID NO: 19; AY029068); nitroreductase (SEQ ID NO: 20; A23284); carboxypeptidase A (SEQ ID NO: 21; M27717); linamarase (SEQ ID NO: 22; S35175); E. coli gpt (SEQ ID NO: 23; X00221); E. coli Deo (SEQ ID NO: 24; X03224); p53 (SEQ ID NO: 25; AF307851); Rb (SEQ ID NO: 26; XM_{—}053409); p15 (SEQ ID NO: 27; U19796); p16 [(SEQ ID NO: 28; U12818) (SEQ ID NO: 29; U12819) and (SEQ ID NO: 30;U12820)]; p21^{WAF-1} (SEQ ID NO: 31; AF497972); GM-CSF (SEQ ID NO: 32; M10663); TNF α (SEQ ID NO: 33; AY066019); IFN α (SEQ ID NO: 34; M34913); IFN γ (SEQ ID NO: 35; J00219); IL1 (SEQ ID NO: 36; M28983); IL2 (SEQ ID NO: 37; K02056); IL3 (SEQ ID NO: 38; M14743); IL4 (SEQ ID NO: 39; M23442); IL6 (SEQ ID NO: 40; M29150); IL7 (SEQ ID NO: 41; J04156); IL10 (SEQ ID NO: 42; U16720); IL12A (SEQ ID NO: 43; NM_{—}000882); IL12B (SEQ ID NO: 44; NM_{—}002187); and IL15 (SEQ ID NO: 45; U14407).

VI. NUCLEIC ACID-BASED EXPRESSION SYSTEMS

The present invention utilizes nucleic acids as vectors or comprised in a separate vector vehicle, wherein the nucleic acids comprise a therapeutic gene regulated by a Tcf-responsive promoter. In specific embodiments, the nucleic acid construct is utilized as therapy for an individual requiring cancer therapy.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

The present invention utilizes a Tcf-responsive promoter operatively linked to a second promoter. The Tcf-responsive promoter comprises a Tcf binding motif. In a preferred embodiment, the Tcf binding motif is SEQ ID NO: 53. In specific, embodiments, the second promoter is minimal CMV promoter, minimal thymidine kinase promoter, thymidine kinase promoter, minimal c-Fos promoter, c-Fos promoter, E2F promoter, and the like. In a specific embodiment, the second promoter facilitates, enhances, or complements regulation by the Tcf-responsive promoter.

A promoter is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 1 lists non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Atchinson et al., 1986, |
| | 1987; Imler et al., 1987; Weinberger et al., 1984; |
| | Kiledjian et al., 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; |
| | Redondo et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 and | |
| GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh |
| | et al., 1985; Firak et al., 1986; Herr et al., |
| | 1986; Imbra et al., 1986; Kadesch et al., 1986; |
| | Wang et al., 1986; Ondek et al., 1987; Kuhl et al., |
| | 1987; Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; |
| | Hen et al., 1986; Satake et al., 1988; Campbell |
| | and/or Villarreal, 1988 |
| Retroviruses | Kriegler et al., 1982, 1983; Levinson et al., |
| | 1982; Kriegler et al., 1983, 1984a, b, 1988; Bosze |
| | et al., 1986; Miksicek et al., 1986; Celander et al., |
| | 1987; Thiesen et al., 1988; Celander et al., 1988; |
| | Choi et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul |
| | et al., 1987; Spandau et al., 1988; Vannice et al., |
| | 1988 |
| Human | Muesing et al., 1987; Hauber et al., 1988; |
| Immunodeficiency | Jakobovits et al., 1988; Feng et al., 1988; Takebe |
| Virus | et al., 1988; Rosen et al., 1988; Berkhout et al., |
| | 1989; Laspia et al., 1989; Sharp et al., 1989; |
| | Braddock et al., 1989 |
| Cytomegalovirus | Weber et al., 1984; Boshart et al., 1985; Foecking |
| (CMV) | et al., 1986 |
| Gibbon Ape | Holbrook et al., 1987; Quinn et al., 1989 |
| Leukemia Virus | |

TABLE 2
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV | Glucocorticoids | Huang et al., 1981; Lee et al., |
| (mouse mammary | | 1981; Majors et al., 1983; |
| tumor virus) | | Chandler et al., 1983; Lee et al., |
| | | 1984; Ponta et al., 1985; Sakai |
| | | et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | Hug et al., 1988 |
| | Newcastle Disease | |
| | Virus | |
| GRP78 Gene | A23187 | Resendez et al., 1988 |
| α-2- | IL-6 | Kunz et al., 1989 |
| Macroglobulin | | |
| Vimentin | Serum | Rittling et al., 1989 |
| MHC Class I | Interferon | Blanar et al., 1989 |
| Gene H-2κb | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid | Thyroid Hormone | Chatterjee et al., 1989 |
| Stimulating | | |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated h erein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the. SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Patent No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippeetal., 1990); by using DEAE-dextran followed by polyethylene glycol (Gopal, 1985); by direct sonic loading (Fechheimeretal., 1987); by liposome mediated transfection (Nicolau and Sene, 1982; Fraleyetal., 1979; Nicolauetal., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh etal., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

a. Ex Vivo Transformation

Methods for tranfecting vascular cells and tissues removed from an organism in an ex vivo setting are known to those of skill in the art. For example, cannine endothelial cells have been genetically altered by retrovial gene tranfer in vitro and transplanted into a canine (Wilson et al., 1989). In another example, yucatan minipig endothelial cells were tranfected by retrovirus in vitro and transplated into an artery using a double-ballonw catheter (Nabel et al., 1989). Thus, it is contemplated that cells or tissues may be removed and tranfected ex vivo using the nucleic acids of the present invention. In particular aspects, the transplanted cells or tissues may be placed into an organism. In preferred facets, a nucleic acid is expressed in the transplated cells or tissues.

b. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of construct comprising a Tcf-responsive promoter regulating a therapeutic gene used may vary upon the nature of the antigen as well as the organelle, cell, tissue or organism used

c. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (Lazerri, 1995), sorghum (Battraw et al., 1991), maize (Bhattachailee et al., 1997), wheat (He et al., 1994) and tomato (Tsukada, 1989).

d. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

e. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

f. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

g. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of- a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

h. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a vector comprising a Tcf-responsive promoter directing expression of a therapeutic gene. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expressioninclude, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. No. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of (expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell

Docket No. AH-UTSC:752US homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

5. Proteins, Polypeptides, and Peptides

The present invention also provides purified, and in preferred embodiments, substantially purified, proteins, polypeptides, or peptides. The term “purified proteins, polypeptides, or peptides” as used herein, is intended to refer to an proteinaceous composition, isolatable from mammalian cells or recombinant host cells, wherein the at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases, which are well known in the art. The coding regions for these known genes may be amplified and/or expressed using the techniques disclosed herein or by any technique that would be know to those of ordinary skill in the art. Additionally, peptide sequences may be sythesized by methods known to those of ordinary skill in the art, such as peptide synthesis using automated peptide synthesis machines, such as those available from Applied Biosystems (Foster City, Calif.).

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. As the present invention provides DNA sequences for the specific proteins, any fusion protein purification method can now be practiced. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

VII. METHODS OF GENE TRANSFER

The present invention addresses compositions and methods utilizing those compositions for the treatment of cancers related to a defective β-catenin/Tcf pathway, wherein the compositions comprise a vector having a therapeutic gene regulated by a Tcf-responsive promoter. The following section describes different vectors that may be utilized for such compositions and methods. In a preferred embodiment, the vector is an adenoviral vector.

A. Adenoviral Vectors

A particular method for delivery of the expression constructs for the determination of β-catenin activation involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and/or (b) to ultimately express a tissue and/or cell-specific construct that has been cloned therein.

The expression vector comprises a genetically engineered form of adenovirus. Knowledge of the genetic organization and/or adenovirus, a 36 kb, linear, double-stranded DNA virus, allows substitution of large pieces of adenoviral DNA with foreign sequences up to 7 kb (Grunhaus and/or Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and/or no genome rearrangement has been detected after extensive amplification.

Adenovirus is particularly suitable for use as a gene transfer vector because of its mid-sized genome, ease of manipulation, high titer, wide target-cell range and/or high infectivity. Both ends of the viral genome contain 100-200 base pair inverted repeats (ITRs), which are cis elements necessary for viral DNA replication and/or packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and/or E2B) results in the synthesis of the proteins for viral DNA replication. These proteins are involved in DNA replication, late gene expression and/or host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and/or all the mRNAs issued from this promoter possess a 5′-tripartite leader (TPL) sequence which makes them preferred mRNA's for translation.

In a current system, recombinant adenovirus is generated from homologous recombination between shuttle vector and/or provirus vector. Due to the possible recombination between two proviral vectors, wild-type adenovirus may be generated from this process. Therefore, it is critical to isolate a single clone of virus from an individual plaque and/or examine its genomic structure.

Generation and/or propagation of the current adenovirus vectors, which are replication deficient, depend on a unique helper cell line, designated 293, which was transformed from human embryonic kidney cells by Ad5 DNA fragments and/or constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 and/or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from human cells such as embryonic kidney cells, muscle cells, hematopoietic cells and/or other embryonic mesenchymal and/or epithelial cells. Alternatively, the helper cells may be derived from the cells of other mammalian species that are permissive for human adenovirus. Such cells include, e.g., Vero cells and/or other monkey embryonic mesenchymal and/or epithelial cells.

Recently, Racher et al. (1995) disclosed improved methods for culturing 293 cells and/or propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, and/or at least conditionally defective, the nature of the adenovirus vector is not believed to be crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and/or genetic information is known, and/or it has historically been used for most constructions employing adenovirus as a vector.

As stated above, the typical vector according to the present invention is replication defective and/or will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and/or in the E4 region where a helper cell line and/or helper virus complements the E4 defect.

Adenovirus growth and/or manipulation is known to those of skill in the art, and/or exhibits broad host range in vitro and/or in vivo. This group of viruses can be obtained in high titers, e.g., 10⁹ to 10¹¹ plaque-forming units per ml, and/or they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991a; Stratford-Perricaudet et al., 1991b; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and/or transduce non-dividing hyman primary cells.

B. AAV Vectors

Adeno-associated virus (AAV) is an attractive vector system for use in the cell transduction of the present invention as it has a high frequency of integration and/or it can infect nondividing cells, thus making it useful for delivery of genes into mammalian cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and/or U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and/or Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994).

AAV is a dependent parvovirus in that it requires coinfection with another virus (either adenovirus and a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome and/or from a recombinant plasmid, and a normal productive infection is established (Samulski et al., 1989; McLaughlin et al., 1988; Kotin et a/, 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and/or an expression plasmid containing the wild type AAV coding sequences without the terminal repeats, for example pIM45 (McCarty et al., 1991; incorporated herein by reference). The cells are also infected and/or transfected with adenovirus and/or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions and/or cell lines containing the AAV coding regions and/or some and/or all of the adenovirus helper genes could be used (Yang et al., 1994; Clark etal., 1995). Cell lines carrying the rAAV DNA as an integrated provirus can also be used (Flotte et al., 1995).

C. Retroviral Vectors

Retroviruses can be gene delivery vectors due to their ability to integrate their genes into the host genome, transferring a large amount of foreign genetic material, infecting a broad spectrum of species and/or cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain. viral sequences to produce a virus that is replication-defective. In order to produce virions, a packaging cell line containing the gag, pol, and/or env genes but without the LTR and/or packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and/or Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected cells bearing the erythropoietin (EPO) receptor. This was achieved by inserting a portion of the EPO sequence into an envelope protein to create a chimeric protein with a new binding specificity.

D. Herpesvirus

Because herpes simplex virus (HSV) is neurotropic, it has generated considerable interest in treating nervous system disorders. Moreover, the ability of HSV to establish latent infections in non-dividing neuronal cells without integrating in to the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than. in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see (Glorioso et al., 1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotide reductase, a DNA polymnerase, a ssDNA binding protein, a helicase/primase, a DNA dependent ATPase, dUTPase and others.

HSV genes from several groups whose expression is coordinately regulated and sequentially ordered in a cascade fashion (Honess and Roizman, 1974; Honess and Roizman, 1975; Roizman and Sears, 1995). The expression of α genes, the first set of genes to be expressed after infection, is enhanced by the virion protein number 16, or α-transducing factor (Post et al., 1981; Batterson and Roizman, 1983; Campbell et al., 1983). The expression of β genes requires functional a gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

E. Lentiviral Vectors

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997), U.S. Pat. Nos. 6,013,516 and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus. enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc. and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

F. Vaccinia Virus

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

G. Polyoma viruses

The empty capsids of papovaviruses, such as the mouse polyoma virus, have received attention as possible vectors for gene transfer (Barr et al., 1979), first described the use of polyoma empty when polyoma DNA and purified empty capsids were incubated in a cell-free system. The DNA of the new particle was protected from the action of pancreatic DNase. Slilaty and Aposhian (1983) described the use of those reconstituted particles for transferring a transforming polyoma DNA fragment to rat FIII cells. The empty capsids and reconstituted particles consist of all three of the polyoma capsid antigens VP1, VP2 and VP3 and there is no suggestion that pseudocapsids consisting of only the major capsid antigen VP1, could be used in genetic transfer.

(Montross et al., 1991), described only the major capsid antigen, the cloning of the polyoma virus VP1 gene and its expression in insect cells. Self-assembly of empty pseudocapsids consisting of VP1 is disclosed, and pseudocapsids are said not to contain DNA. It is also reported that DNA inhibits the in vitro assembly of VP1 into empty pseudocapsids, which suggests that said pseudocapsids could not be used to package exogenous DNA for transfer to host cells. The results of (Sandig et al., 1993), showed that empty capsids incorporating exogenous DNA could transfer DNA in a biologically functional manner to host cells only if the particles consisted of all three polyoma capsid antigens VP1, VP2 and VP3. Pseudocapsids consisting of VP1 were said to be unable to transfer to exogenous DNA so that it could be expressed in the host cells, probably due the absence of Ca²⁺ ions in the medium in which the pseudocapsids were prepared. Haynes et al (1993) discuss the effect of calcium ions on empty VP1 pseudocapsid assembly.

U.S. Pat. No. 6,046,173, issued on Apr. 4, 2000, and entitled “Polyoma virus pseudocapsids and method to deliver material into cell,” reports on the use of a pseudocapsid formed from papovavirus major capsid antigen and excluding minor capsid antigens, which pseudocapsid incorporates exogenous material for gene transfer.

H. Other Viral Vectors

Other viral vectors may be employed as expression constructs in the present invention. Vectors derived from viruses such as sindbis virus and/or cytomegalovirus. They offer several attractive features for various mammalian cells (Friedmann, 1989; Ridgeway, 1988; Baichwal and/or Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and/or reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

I. Modified Viruses

In still further embodiments of the present invention, the nucleic acids to be delivered are housed within an infective virus that has been engineered to express a specific binding ligand. The virus particle will thus bind specifically to the cognate receptors of the target cell and/or deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and/or against a specific cell receptor were used. The antibodies were coupled via the biotin components by using streptavidin (Roux et al., 1989). Using antibodies against major histocompatibility complex class I and/or class II antigens, they demonstrated the infection of a variety of human cells that bore those surface antigens with an ecotropic virus in vitro (Roux et al., 1989).

VIII. LIPID COMPOSITIONS

In certain embodiments, the present invention concerns a novel composition comprising one or more lipids associated with at least one composition as described herein. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

A. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about.26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.ka. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a-carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

B. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

C. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

D. Lipid Composition Structures

In a preferred embodiment of the invention, the compositions descirbed herein may be associated with a lipid. A construct comprising aTcf-responsive promoter regulating a therapeutic gene associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/construct comprising aTcf-responsive promoter regulating a therapeutic gene associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-construct comprising aTcf-responsive promoter regulating a therapeutic gene or Superfect (Qiagen)-construct comprising aTcf-responsive promoter regulating a therapeutic gene complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 19%to about 35% phosphatidyl choline, and about 1% of a drug. Thus, it is contemplated that lipid compositions of the present invention may comprise any of the lipids, lipid types or other components in any combination or percentage range.

1. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

2. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

E. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In certain less preferred embodiments, phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition or a liposome, because of the instability and leakiness of the resulting liposomes.

In particular embodiments, a lipid and/or construct comprising aTcf-responsive promoter regulating a therapeutic gene may be, for example, encapsulated in the aqueous interior of a liposome, interspersed within the lipid bilayer of a liposome, attached to a liposome via a linking molecule that is associated with both the liposome and the construct comprising aTcf-responsive promoter regulating a therapeutic gene, entrapped in a liposome, complexed with a liposome, etc.

1. Making Liposomes

A liposome used according to the present invention can be made by different methods, as would be known to one of ordinary skill in the art. Phospholipids can form a variety of structures other than liposomes when dispersed in water, depending on the molar ratio of lipid to water. At low ratios the liposome is the preferred structure.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the construct comprising aTcf-responsive promoter regulating a therapeutic gene, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the construct comprising aTcf-responsive promoter regulating a therapeutic gene is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/construct comprising aTcf-responsive promoter regulating a therapeutic gene or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs. Liposomal encapsulation has resulted in a lower toxicity and a longer serum half-life for such compounds (Gabizon et al., 1990).

Liposomes interact with cells to deliver agents via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

2. Liposome Targeting

Association of the construct comprising aTcf-responsive promoter regulating a therapeutic gene with a liposome may improve biodistribution and other properties of the construct comprising aTcf-responsive promoter regulating a therapeutic gene. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/construct comprising aTcf-responsive promoter regulating a therapeutic gene composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of a construct comprising aTcf-responsive promoter regulating a therapeutic gene. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

a. Cross-linkers

Bifunctional cross-linking reagents have been extensively used for a variety of purposes including preparation of affinity matrices, modification and stabilization of diverse structures, identification of ligand and receptor binding sites, and structural studies. Homobiffunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 3 details certain hetero-bifunctional cross-linkers considered useful in the present invention.

TABLE 3
HETERO-BIFUNCTIONAL CROSS-LINKERS

| | | | Spacer Arm |
|---|---|---|---|
| | | | Length\after |
| Linker | Reactive Toward | Advantages and Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

In instances where a particular polypeptide does not contain a residue amenable for a given cross-linking reagent in its native sequence, conservative genetic or synthetic amino acid changes in the primary sequence can be utilized.

b. Targeting Ligands

The targeting ligand can be either anchored in the hydrophobic portion of the complex or attached to reactive terminal groups of the hydrophilic portion of the complex. The targeting ligand can be attached to the lipQsome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., Chem. Phys. Lipids 40:347 (1986)) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a construct comprising aTcf-responsive promoter regulating a therapeutic gene may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific construct comprising aTcf-responsive promoter regulating a therapeutic gene delivery and/or targeting vehicle may comprise a specific binding ligand in combination with a liposome. The construct comprising aTcf-responsive promoter regulating a therapeutic gene to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and a construct comprising aTcf-responsive promoter regulating a therapeutic gene-binding agent. Others comprise a cell receptor-specific ligand to which construct comprising aTcf-responsive promoter regulating a therapeutic gene to be delivered has been operatively attached. For example, several ligands have been used for receptor-mediated gene transfer (Wu and Wu, 1987; Wagner et al., 1990; Perales et al., 1994;. Myers, EPO 0273085), which establishes the operability of the technique. In another example, specific delivery in the. context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolauetal., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

3. Liposome/Nucleic Acid Combinations

In certain embodiments, a liposome/construct comprising a Tcf-responsive promoter regulating a therapeutic gene may comprise a nucleic acid, such as, for example, an oligonucleotide, a polynucleotide or a nucleic acid construct (e.g., an expression vector). Where a bacterial promoter is employed in the DNA construct that is to be transfected into eukaryotic cells, it also will be desirable to include within the liposome an appropriate bacterial polymerase.

It is contemplated that when the liposome/construct comprising a Tcf-responsive promoter regulating a therapeutic gene composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4orf4 and the E.coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

4. Lipid Administration

The actual dosage amount of a lipid composition (e.g., a liposome-construct comprising a Tcf-responsive promoter regulating a therapeutic gene) administered to a patient can be .determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally,topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage.

5. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

6. Specific Embodiments for Lipid-Based Gene Delivery

Liposomes, micelles, and lipid dispersions can be prepared using any of a variety of lipid components (and potentially other components) that can be complexed with nucleic acid or which can entrap e.g., an aqueous compartment comprising a nucleic acid. Illustrative molecules that can be employed include phosphatidylcholine (PC), phosphatidylserine (PS), cholesterol (Chol), N-[1-(2,3-dioleyloxy)propyl]-N,N-trimethylammonium chloride (DOTMA), dioleoylphosphatidylethanolamine (DOPE), and/or 3β[N-(N′,N′-dimethylamino-ethane)-carbarmoyl cholesterol (DC-Chol), as well as other lipids known to those of skill in the art. Those of skill in the art will recognize that there are a variety of lipid-based transfection techniques which will be useful in the present invention. Among these techniques are those described in Nicolau et al., 1987, Nabel et al., 1990, and Gao et al., 1991. The inventors have had particular success with lipid/DNA complexes comprising DC-Chol. More particularly, the inventors have had success with lipid/DNA complexes comprising DC-Chol and DOPE which have been prepared following the teachings of L. Huang and collaborators (see, e.g., Gao et al., 1991; Epand et al., PCT/US92/07290, and U.S. Pat. No. 5,283,185). Lipid complexes comprising DOTMA, such as those which are available commercially under the trademark Lipofectin™, from Vical Inc., San Diego, Calif., may also be used. A variety of improved techniques for lipid-based gene delivery that can be employed to deliver genes such as those disclosed herein have been described by L. Huang and collaborators (Deshmukh et al., PCT/US97/06066; Liu et al., PCT/US96/15388, and Huang et al., PCT/US97/12544).

Lipid/nucleic acid complexes can be introduced into contact with cells to be transfected by a variety of methods. In cell culture, the complexes can simply be dispersed in the cell culture solution. For application in vivo, the complexes are typically injected. Intravenous injection allows lipid-mediated transfer of complexed DNA to, for example, the liver and the spleen. In order to allow transfection of DNA into cells which are not accessible through intravenous injection, it is possible to directly inject the lipid-DNA complexes into a specific location in an animal's body. For example, Nabel et al. teach injection of liposomes via a catheter into the arterial wall. In another example, the present inventors have used intraperitoneal injection of lipid/DNA complexes to allow for gene transfer into mice.

The present invention also contemplates compositions comprnsing a lipid complex. This lipid complex will generally comprise a lipid component and a composition as described herein.

The lipid employed to make the lipid complex can be any of the above-discussed lipids. In particular, DOTMA, DOPE, and/or DC-Chol may form all or part of the lipid complex. The inventors have had particular success with complexes comprising DC-Chol. In a preferred embodiment, the lipid complex comprises DC-Chol and DOPE. While many ratios of DC-Chol to DOPE can have utility, it is anticipated that those comprising a ratio of DC-Chol:DOPE between 1:20 and 20:1 will be particularly advantageous. The inventors have found that lipid complexes prepared from a ratio of DC-Chol:DOPE of about 1:10 to about 1:5 have been particularly useful from the standpoint of stability as well as efficacy. Lipid and liposomes that may be used in conjunction with delivery of compositions described herein are also described in U.S. Pat. Nos. 5,922,688, 5,814,315, 5,651,964, 5,641,484, and 5,643,567, the entire texts of each being specifically incorporated herein by reference; also see pending U.S. patent application Ser. No. 08/809,021, filed Mar. 19, 1998, also incorporated herein by reference.

In certain embodiments of the invention, the lipid may also be complexed with a hemagglutinating virus (HVJ). This has been shown to facilitate fusion with the cell membrane and to promote cell entry of liposome-encapsulated DNA (Kaneda et al., 1989). In other embodiments, the lipid may be complexed or employed in conjunction with nuclear non-histone chromosomal proteins ( MG-1) (Kato et al., 1991). In yet further embodiments, the lipid may be complexed or employed in conjunction with both HVJ and HMG-1. Work by Huang and collaborators has also provided a number of lipid-based gene delivery compositions, some comprising nucleic acid condensing agents and other components; and has further described detailed techniques that can be used for the production of such gene delivery complexes (see, e.g., Targeted Genetics Corporation PCT/US97/12544 as well as other references by Huang et al. above).

In that such expression constructs have been successfully employed in transfer and expression of nucleic acid in vitro and in vivo, then they are applicable for the present invention. As is known in the art, one can also include other components within the gene delivery complex, including proteins and/or other molecules that facilitate targeting to particular cells, binding and uptake by targeted cells, localization within particular subcellular compartments (e.g., the nucleus or cytosol), as well as integration and/or expression of the DNA delivered. A variety of such individual components, and combinations thereof, have been described by Targeted Genetics Corporation in PCT/US95/04738.

IX. METHODS TO IDENTIFY β-CATENIN ACTIVATION

In still further embodiments, the present invention provides methods for identifying whether the activation of β-catenin has been altered, such as for identification of a cancer cell to be treated. The changes in β-catenin activation can be determined by observing the localization of β-catenin at different locations in the cell. β-catenin localization at the cell cytoplasm or cell nucleus is described as activation of β-catenin where localization of β-catenin at the plasma membrane is described as a decrease in β-catenin activation. It is contemplated that a variety of techniques can be used to obtain β-catenin activation.

A. Immunoassays

Immunodetection methods may be used in the current invention for detecting, binding, purifying, removing and quantifying the proteins and peptides of the current invention. The proteins or peptides of the present invention may be employed to detect antibodies having reactivity therewith, or, alternatively, antibodies prepared in accordance with the present invention, may be employed to detect activation of β-catenin.

The steps of various useful immunodetection methods have been described in the scientific literature, such as, e.g., Nakamura et al. (1987; incorporated herein by reference). Immunoassays, in their most simple and-direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods of this invention include methods for detecting or quantifying the amount of a reactive component in a sample, which methods require the detection or quantitation of any immune complexes formed during the binding process. Here, one would obtain a β-catenin protein, peptide or a corresponding antibody, and contact it with an antibody or protein or peptide, as the case may be, and then detect or quantify the amount of immune complexes formed under the specific conditions.

In terms of antigen detection, the biological sample analyzed may be any sample that is suspected of containing an antigen specific to the cell adhesion proteins or cyclin D1 of the current invention. The sample can be a tissue section or specimen, a homogenized tissue extract, an isolated cell, a cell membrane preparation, separated or purified forms of any of the above protein-containing compositions, or even any biological fluid that comes into contact with tissue such as blood. Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. Pat. Nos. concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The protein, peptide or corresponding antibody employed in the detection may itself be linked to a detectable label, wherein one would then simply detect this label, thereby allowing the amount of the primary immune complexes in the composition to be determined. Epitope tags are useful for the labeling and detection of proteins using immunoblotting, immunoprecipitation and immunostaining techniques. Due to their small size, they are unlikely to affect the tagged protein's biochemical properties. The Myc epitope tag is widely used to detect expression of recombinant proteins in bacteria, yeast, insect and mammalian cell systems (Munro et al, 1984).

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

B. Western Blot

It is contemplated that the methods of the current invention can include Western Blot analysis. Western Blot analysis can be used to determine the effectiveness of, for example, the up-regulation of cyclin D1 promoter activity and protein expression by β-catenin. Preferred detection methods include chemiluminescence and chromagenic detection. Standard methods for Western Blot analysis can be found in, for example, Bollag et al., 1996 or Harlow et al. 1988, herein incorporated by reference.

C. ELISAs

As noted, it is contemplated that the ELISA may be used to study the regulation of cyclin D1 promoter activity and protein expression by β-catenin.

In one exemplary ELISA, antibodies binding to the proteins of the invention are immobilized onto a selected surface exhibiting protein affinity, such as a well in a polystyrene microtiter plate. Then, a test composition suspected of containing a marker antigen is added to the wells. After binding and washing to remove non-specifically bound immunocomplexes, the bound antigen may be detected.

Detection is generally achieved by the addition of a second antibody specific for the target protein, that is linked to a detectable label. This type of ELISA is a simple “sandwich ELISA”. Detection also may be achieved by the addition of a second antibody, followed by the addition of a third antibody that has binding affinity for the second antibody, with the third antibody being linked to a detectable label.

In another exemplary ELISA, a marker antigen is immobilized onto the well surface and then contacted with the antibodies of the invention. After binding and washing to remove non-specifically bound immunecomplexes, the bound antibody is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides, are immobilized, involves the use of antibody competition in the detection. In this ELISA, labeled antibodies are added to the wells, allowed to bind to the marker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

Under conditions effective to allow immunecomplex (antigen/antibody) formation means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the fonnation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for. a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

In other embodiments, solution -phase competition ELISA is also contemplated. Solution phase ELISA involves attachment of a protein a related peptide to a bead, for example a magnetic bead. The bead is then incubated with sera from human and animal origin. After a suitable incubation period to allow for specific interactions to occur, the beads are washed. The specific type of antibody is the detected with an antibody indicator conjugate. The beads are washed and sorted. This complex is the read on an appropriate instrument (fluorescent, electroluminescent, spectrophotometer, depending on the conjugating moiety). The level of antibody binding can thus by quantitated and is directly related to the amount of signal present.

D. Immunohistochemistry

The proteins and antibodies of the present invention may also be used in conjunction with both fresh-frozen and formalin-fixed, paraffin-embedded tissue blocks prepared for study by immunohistochemistry (IHC). For example, each tissue block consists of 50 mg of residual “pulverized” breast tumor tissue. The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” breast tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections maybe prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

E. FACS Analyses

Fluorescent activated cell sorting, flow cytometry or flow microfluorometry provides the means of scanning individual cells for the presence of activated or non-activated β-catenin. The method employs instrumentation that is capable of activating, and detecting the excitation emissions of labeled cells in a liquid medium. FACS is unique in its ability to provide a rapid, reliable, quantitative, and multiparameter analysis on either living or fixed cells. The antibodies of the present invention provide a useful tool for the analysis and quantitation of markers of individual cells.

F. Gel-shift Assay

The gel shift assay or electrophoretic mobility shift assay (EMSA) is used to detect the interactions between DNA binding proteins and their cognate DNA recognition sequences, in both a qualitative and quantitative manner. The technique was originally developed for DNA binding proteins, but has since been extended to allow detection of RNA binding proteins due to their interaction with a particular RNA sequence.

In a general gel-shift assay, purified proteins or crude cell extracts are incubated with a 32P-radiolabeled DNA or RNA probe, followed by separation of the complexes from the free probe through a nondenaturing polyacrylamide gel. The complexes will migrate more slowly through the gel than unbound probe. Depending on the activity of the binding protein, a radiolabeled probe may be either double-stranded or single-stranded. For the detection of DNA binding proteins such as transcription factors, either purified or partially purified proteins, or nuclear cell extracts are used. For detection of RNA binding proteins, either purified or partially purified proteins, or nuclear or cytoplasmic cell extracts are used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. (http://www.shpromega.com.cn/gelshfaq.html#q01)

G. In vivo Imaging

The invention also provides in vivo methods of imaging β-catenin activation using antibody conjugates. The term “in vivo imaging” refers to any non-invasive method that permits the detection of a labeled antibody, or fragment thereof, that specifically binds to cancer cells located in the body of an animal or human subject.

The imaging methods generally involve administering to an animal or subject an imaging-effective amount of a detectably-labeled fcatenin, cyclin D1 or α-actin specific antibody or fragment thereof (in a pharmaceutically effective carrier), such as an antibody to β-catenin, cyclin D1 or α-actin, and then detecting the location of the labeled antibody in the sample cell. The detectable label is preferably a spin-labeled molecule or a radioactive isotope that is detectable by non-invasive methods.

An “imaging effective a-mount” is an amount of a detectably-labeled antibody, or fragment thereof, that when administered is sufficient to enable later detection of binding of the antibody or fragment to cancer tissue. The effective amount of the antibody-marker conjugate is allowed sufficient time to come into contact with reactive antigens that be present within the tissues of the patient, and the patient is then exposed to a detection device to identify the detectable marker.

Antibody conjugates or constructs for imaging thus have the ability to provide an image of the tumor, for example, through magnetic resonance imaging, x-ray imaging, computerized emission tomography and the like. Elements particularly useful in Magnetic Resonance Imaging (“MRT”) include the nuclear magnetic spin-resonance isotopes ¹⁵⁷Gd, ⁵⁵Mn, ¹⁶²Dy, ⁵²Cr, and ⁵⁶Fe, with gadolinium often being preferred. Radioactive substances, such as technicium^{99m} or indium¹¹¹, that may be detected using a gamma scintillation camera or detector, also may be used. Further examples of metallic ions suitable for use in this invention are ¹²³I, ¹³¹I, ¹³¹I, ⁹⁷Ru, ⁶⁷Cu, ⁶⁷Ga, ¹²⁵I, ⁶⁸Ga, ⁷²As, ⁸⁹Zr, and ²⁰¹Tl.

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

A radionuclide may be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA).

Administration of the labeled antibody may be local or systemic and accomplished intravenously, intra-arterially, via the spinal fluid or the like. Administration also may be intradermal or intracavitary, depending upon the body site under examination. After a sufficient time has lapsed for the labeled antibody or fragment to bind to the diseased tissue, in this case cancer tissue, for example 30 min to 48 h, the area of the subject under investigation is then examined by the imaging technique. MRI, SPECT, planar scintillation imaging and other emerging imaging techniques may all be used.

The distribution of the bound radioactive isotope and its increase or decrease with time is monitored and recorded. By comparing the results with data obtained from studies of clinically normal individuals, the presence and extent of the diseased tissue can be determined.

The exact imaging protocol will necessarily vary depending upon factors specific to the patient, and depending upon the body site under examination, method of administration, type of label used and the like. The determination of specific procedures is, however, routine to the skilled artisan. Although dosages for imaging embodiments are dependent upon the age and weight of patient, a one time dose of about 0.1 to about 20 mg, more preferably, about 1.0 to about 2.0 mg of antibody-conjugate per patient is contemplated to be useful.

X. SCREENING FOR MODULATORS OF β-CATENIN TRANSCRIPTIONAL Activity

In cells having an increase in b-catenin activity, often the cell is cancerous. Therefore, it is useful to provide a means to identify compositions that can decrease or quench such an increase in activity. The present invention provides methods of screening for modulators, e.g., inhibitors, of β-catenin activity. Such modulators would be useful to alter β-catenin activity in a patient, for the treatment of a number of cancers. Thus, the invention provides assays for β-catenin modulation, where the compositions described herein facilitate identification of an inhibitor of β-catenin activity.

“Inhibitors,” “activators,” and “modulators” of β-catenin refer to any inhibitory molecules identified using in vitro and in vivo assays for β-catenin, e.g., antagonists, and their homologs and mimetics, using the vectors described herein. Inhibitors are compounds that decrease, block, prevent, delay activation, inactivate, desensitize, or down regulate β-catenin, e.g., antagonists. Modulators include genetically-modified versions of β-catenin, e.g., with altered activity, as well as naturally-occurring and synthetic ligands, antagonists, agonists, small chemical molecules and the like. Samples or assays comprising β-catenin that are treated with a potential activator, inhibitor, or modulator are compared to control samples without the inhibitor, activator, or modulator to examine the extent of inhibition. Control samples (untreated with inhibitors) are assigned a relative β-catenin activity value of 100%. Inhibition of β-catenin, or blocking the pathway to form β-catenin is achieved when the β-catenin activity value relative to the control is about 80%, preferably 50%, more preferably 25−1%. Activation of β-catenins is achieved when the β-catenin activity value relative to the control is 110%, more preferably 150%, more preferably 200-500%, more preferably 1000-3000% higher.

In numerous embodiments of this invention, assays will be performed to detect compounds that affect β-catenin activity. Such assays can involve the identification of compounds that interact with β-catenin proteins, either physically or genetically, and can thus rely on any of a number of standard methods to detect physical or genetic interactions between compounds.

In specific embodiments, a Tcf-responsive promoter (such as comprising a minimal CMV promoter)-driven reporter gene is used to screen for drugs inhibiting the transcriptional function of β-catenin. A skilled artisan is aware of a variety of reporter genes that may be utilized, including green fluorescent protein (GFP), blue fluorescent protein (BFP), β-galactosidase, luciferase, chloramphenicol acetyl transferase, and the like.

In certain embodiments, assays will be performed to identify molecules that interact with a Tcf-responsive promoter. The interaction may be direct or indirect. Such molecules can be any type of molecule, including polypeptides, polynucleotides, amino acids, nucleotides, carbohydrates, lipids, or any other organic or inorganic molecule. Such molecules may represent molecules that normally interact with a Tcf-responsive promoter to effect regulation of an endogenously regulated a Tcf-responsive promoter. Alternatively, they may be synthetic or other molecules that are capable of interacting with a Tcf-responsive promoter and which can potentially be used to modulate β-catenin activity in cells, or used as lead compounds to identify classes of molecules that can interact with and/or modulate β-catenin.

In a particular embodiment, the method of screening for a modifier of β-catenin activity comprises providing a β-catenin/Tcf-responsive promoter construct comprising a first promoter region having at least one copy of a Tcf/LEF-1 binding site, operatively linked to; a second promoter; and a reporter nucleic acid sequence, wherein the first and second promoter regions are operatively linked to the reporter nucleic acid sequence. A test compound is introduced to the vector, and a change associated with the reporter nucleic acid sequence is assayed. When a change occurs, the test compound is the modifier. In the embodiment wherein the transcription rate or level decreases, the modifier is an inhibitor of β-catenin activity.

A skilled artisan recognizes that the inhibitors identified by the screening methods described herein are useful for the treatment of cancers related to β-catenin/Tcf pathway. In a specific embodiment, the inhibitors identified by methods described herein are combined with a pharmaceutical carrier and administered to a patient having a cancer related to the β-catenin/Tcf pathway.

XI. CANCER TREATMENT

The present invention regards therapy for cancer patients directed to a Tcf-responsive promoter construct regulating a therapeutic gene. Furthermore, the screening methods described above preferably identify a composition for therapeutic administration to a person with cancer, optionally in combination with an effective amount of a second agent, for example a chemotherapeutic agent or any other anti-cancer agent are contemplated. These modulators include genetically-modified versions of β-catenin, e.g., with altered activity, as well as naturally-occurring and synthetic ligands, antagonists, small chemical molecules and the like.

For the sake of brevity, the cancer treatments described herein directed to administration of a construct comprising a Tcf-responsive promoter regulating a therapeutic gene and the treatments regarding inhibitors of β-catenin activity for a cancer treatment as identified by a screen using a Tcf-responsive promoter regulating a reporter gene will be hereafter collectively referred to as Tcf-responsive promoter-related therapies.

A. Pharmaceutical Compositions

The Tcf-responsive promoter-related compositions will generally be dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti -cancer agents, can also be incorporated into the compositions.

B. Routes of Administration

Tcf-responsive promoter-related compounds may be formulated for parenteral administration as well for as other administration methods such as intravenous, intramuscular or intratumoral injection, other pharmaceutically acceptable forms include, e.g., tablets or other solids for oral administration; time release capsules; and any other form currently used, including cremes, lotions, rinses, inhalants and the like.

The expression vectors and delivery vehicles of the present invention may include classic pharmaceutical preparations. Administration of these compositions according to the present invention will be via any common route so long as the target tissue is available via that route. This includes oral, nasal or topical. Alternatively, administration may be by, e.g., orthotopic, intradermal, subcutaneous, intramuscular, intraperitoneal or intravenous injection. Such compositions would normally be administered as pharmaceutically acceptable compositions, described supra.

The vectors of the present invention are advantageously administered in the form of injectable compositions either as liquid solutions or suspensions. Solid forms suitable for solution in, or suspension in, liquid prior to injection also may be prepared. These preparations also may be emulsified. A typical composition for such purposes comprises 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions can take the form of solutions, suspensions, tablets, pills, capsules, sustained release formulations or powders. When the route is topical, the form may be a cream, ointment, salve or spray.

An effective amount of the Tcf-responsive promoter-related therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

C. Treatment Protocols

The treatment of human cancers using a Tcf-responsive promoter-related composition is contemplated in the current invention. For gene modulators, this may be achieved by introduction of the desired modulator gene through the use of a viral or non-viral vector to carry the therapeutic sequences regulated by the Tcf-responsive promoter to efficiently and specifically infect the tumor, or pre-tumorous tissue. Viral vectors will preferably be an adenoviral, a retroviral, a vaccinia viral vector or adeno-associated virus as described hereinabove (Muro-cacho et al, 1992). These vectors are preferred because they have been successfully used to deliver desired sequences to cells and tend to have a high infection efficiency. Non-viral vectors include liposomes.

Tcf-responsive promoter-related compositions may be administered parenterally or orally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. A Tcf-responsive promoter-related composition may be delivered to the patient before, after or concurrently with the other anti-cancer agents. A typical treatment course may, for example, comprise about six doses delivered over a 7 to 21 day period. Upon election by the clinician the regimen may be continued six doses every three weeks or on a less frequent (monthly, bimonthly, quarterly, etc.) basis. Of course, these are only exemplary times for treatment, and the skilled practitioner will readily recognize that many other time-courses are possible.

Regional delivery of a Tcf-responsive promoter-related composition will be an efficient method for delivering a therapeutically effective dose to counteract the clinical disease. Likewise, the chemotherapy may be directed to a particular affected region. Alternatively, systemic delivery of either, or both, agent may be appropriate. The therapeutic composition of the present invention is administered to the patient directly at the site of the tumor. This is in essence a topical treatment of the surface of the cancer. The volume of the composition should usually be sufficient to ensure that the entire surface of the tumor is contacted by a β-catenin modulator, and second agent. In one embodiment, administration simply entails injection of the therapeutic composition into the tumor. In another embodiment, a catheter is inserted into the site of the tumor and the cavity may be continuously perfused for a desired period of time.

A major challenge in clinical oncology is that many tumor cells are resistant to chemotherapeutic treatment. One goal of the inventors' efforts has been to find ways to improve the efficacy of chemotherapy. In the context of the present invention, a Tcf-responsive promoter-related composition can be combined with any of a number of conventional chemotherapeutic regimens. Patients to be treated with a Tcf-responsive promoter-related composition may, but need not, have received previous surgical, chemo- radio- or gene therapeutic treatments.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

Of course, the above-described treatment regimes may be altered in accordance with the knowledge gained from clinical trials such as those described herein. Those of skill in the art will be able to take the information disclosed in this specification and optimize treatment regimes based on the clinical trials described in the specification.

D. Clinical Trials

Human treatment protocols may be developed using Tcf-responsive promoter-related composition(s), alone or in combination with other anti-cancer drugs. The Tcf-responsive promoter-related composition, and anti-cancer drug treatment will be of use in the clinical treatment of various cancers such as colon cancer. Such treatment will be particularly useful tools in anti-tumor therapy, for example, in treating patients with colon cancers that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the Tcf-responsive promoter-related composition(s) in clinical trials.

Patients with advanced, metastatic colon, breast, epithelial ovarian carcinoma, pancreatic, or other cancers chosen for clinical study will typically have failed to respond to at least one course of conventional therapy.

In regard to the Tcf-responsive promoter-related composition therapy, a Tcf-responsive promoter-related composition may be administered alone or in combination with the other anti-cancer drug. The administration may be directly into the tumor, or in a systemic manner. The starting dose may be anywhere from 0.01 to 5.0 mg/kg body weight. Three patients may be treated at each dose level in the absence of grade>3 toxicity. Dose escalation may be done by 100% increments (e.g. 0.5 mg, 1 mg, 2 mg, 4 mg) until drug related grade 2 toxicity is detected. Thereafter dose escalation may proceed by 25% increments. The administered dose may be fractionated equally into multiple infusions, separated by 1 to 12 hours if the lot of anti-cancer drug exceed 5 EU/kg for any given patient.

The Tcf-responsive promoter-related composition, and/or the other anti-cancer drug combination, may be administered over a short infusion time or at a steady rate of infusion over a 1 to 356 day period. The Tcf-responsive promoter-related composition infusion may be administered alone or in combination with an anti-cancer drug or surgery. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the Tcf-responsive promoter-related composition, in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies can include mammograms, CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum could be monitored.

To monitor disease course and evaluate the anti-tumor responses, it is contemplated that the patients should be examined for appropriate tumor markers every 2-6 weeks, if initially abnormal. Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

X. COMBINATION THERAPIES

In order to increase the effectiveness of the therapy by Tcf-responsive promoter-related compositions as described in the present invention, it may be desirable to combine these compositions with yet other agents effective in the treatment of a cancer, such as colon cancer.

In the context of the present invention, it is therefore contemplated that the Tcf-responsive promoter-related composition therapy will be used in combination with other anticancer-therapies known in the art for treating cancers that have an increased β-catenin activation. A variety of cancers including pre-cancers, tumors, malignant cancers can be treated according to the methods of the present invention. Some of the cancer types contemplated for treatment in the present invention include colon cancer, metastasized colon cancer, such as to the liver, breast, prostate, liver, myelomas, bladder, blood, bone, bone marrow, brain, colon, esophagus, gastrointestine, head, kidney, lung, nasopharynx, neck, ovary, skin, stomach, and uterus cancers. The treatment of colon cancer and/or metastasized colon cancer to the liver is preferred.

The administration of the other anti-cancer therapy or surgical procedure may precede or follow the Tcf-responsive promoter-related composition therapy by intervals ranging from minutes to days to weeks. In embodiments where the other anti-cancer therapy and the Tcf-responsive promoter-related composition therapy are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective admirnistrations.

It also is conceivable that more than one administration of either the other anti-cancer therapy and the Tcf-responsive promoter-related composition will be required in the preferential cancer treatment regime. Various combinations may be employed, where the other anti-cancer therapy agent is “A” and the Tcf-responsive promoter-related composition therapy is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations also are contemplated. The exact dosages and regimens of each agent can be suitable altered by those of ordinary skill in the art.

Some examples of other anti-cancer therapies that may be used include chemotherapeutic agents, surgery, immunotherapy, gene therapy, hormonal therapy, or other anti-cancer therapies. It is also contemplated that other chemotherapeutics may be used, such as but not limited to, cisplatin, gemcitabine, novelbine, doxorubicin, VP16, TNF, emodin, daunorubicin, dactinomycin, mitoxantrone, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil (5FU), camptothecin, actinomycin-D, hydrogen peroxide, nitrosurea, plicomycin, tamoxifen, transplatinum, vincristin, vinblastin, TRAIL, or methotrexate.

XII. KITS

The materials and reagents required for providing therapy for cancers having activation of the Wnt/β-catenin pathway as described herein may be assembled together in a kit. In one embodiment, such a kit generally will comprise vectors as described herein, or fragments thereof. If fragments of vectors are provided, the kit may also comprise means to assemble the fragments, such as ligation enzymes.

In a specific embodiment, the kit comprises a vector having a Tcf-responsive promoter and a second promoter, both of which regulate expression of a therapeutic gene comprised on the vector. In specific embodiments, the vector is a viral vector. In further specific embodiments, the viral vector is an adenoviral vector.

In each case, the kits will preferably comprise distinct containers for each individual reagent. Each biological agent, such as DNA or fragments thereof will generally be suitable aliquoted in their respective containers. The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

When the components of the kit are provided in one or more liquid solutions, the liquid solution preferably is an aqueous solution, with a sterile aqueous solution being particularly preferred. For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs or even applied to and mixed with the other components of the kit. The components of the kit may also be provided in dried or lyophilized forms. When reagents or components are provided as a dried form, reconstitution generally is by the addition of a suitable solvent. It is envisioned that the solvent also may be provided in another container means.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

XIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Analysis of Multiple β-Catenin/Tcf-Responsive Promoters

The present invention is directed to a gene therapy system, particularly for the treatment of cancer. In a specific embodiment, the cancer cells in the individual being treated comprise an activated β-catenin/Tcf pathway. In another specific embodiment, the cancer cells are colon cells. In a further specific embodiment, the cancer cells are metastasized colon cells, such as to the liver. In a preferred embodiment, the compositions and methods described herein are deleterious to cancer cells but do not affect cells that do not have an activated β-catenin/Tcf pathway.

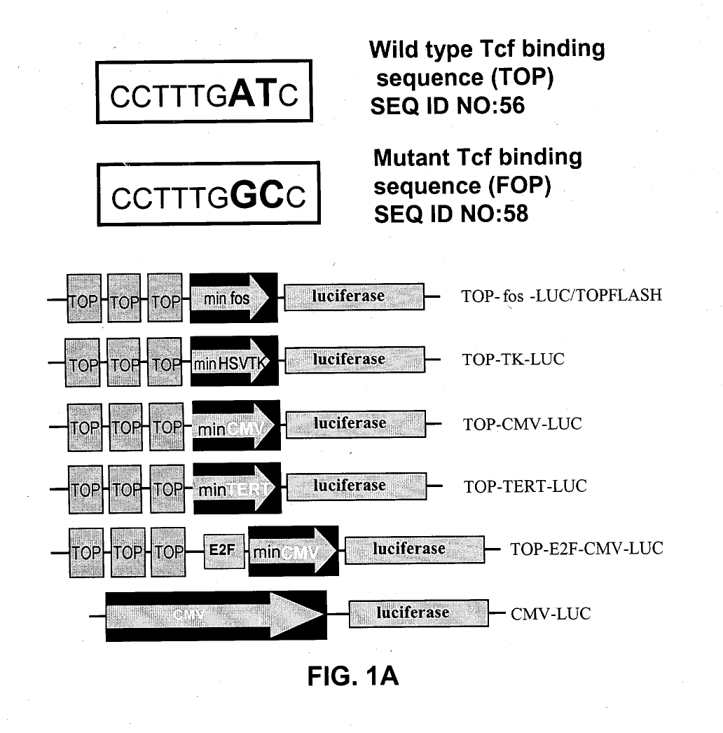

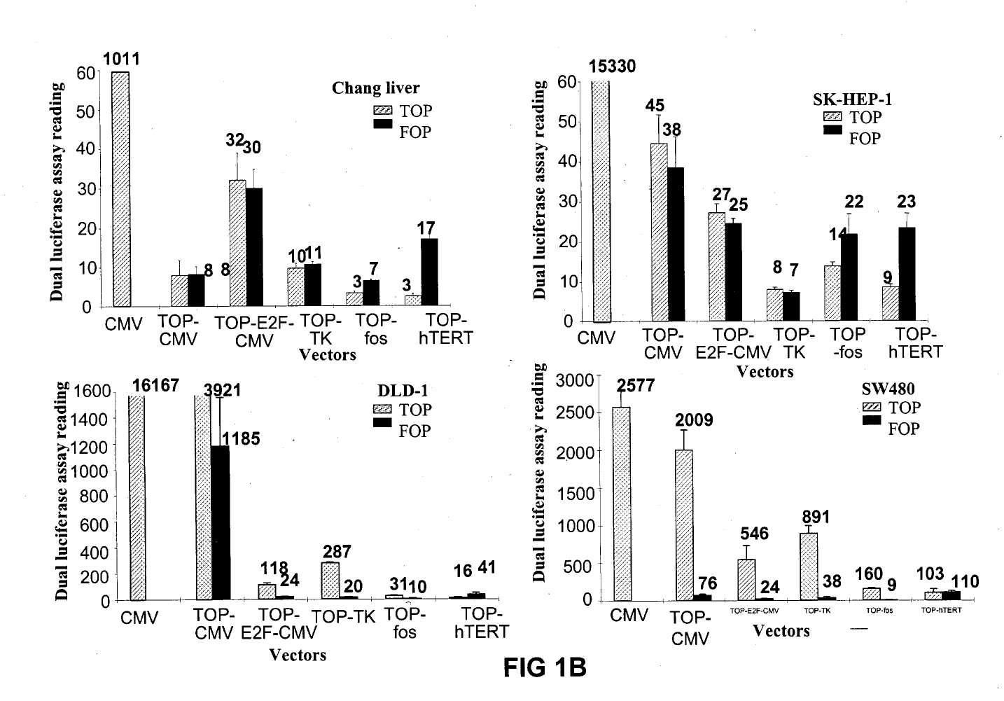

For the gene therapy system, multiple β-catenin/Tcf-responsive promoters that can selectively target colon cancer were analyzed. The activities of five sets of β-catenin/Tcf-responsive promoters were compared in colon cancer cell lines. Two well-characterized colon cancer cell lines, SW480 and DLD-I, were selected. In both of the cell lines, the APC gene is mutated and β-catenin levels are elevated. Chang liver and SK-HEP-1 cell lines were included in this study as controls. These two cell lines are derived from liver origins and exhibit very low level of β-catenin/Tcf transcription activity. FIG. 1A illustrates the structures of the five sets of β-catenin/Tcf responsive promoters. All five promoters contain three copies of β-catenin/Tcf-binding site sequences (wild-type β-catenin/Tcf-binding sites in TOP promoter and mutated β-catenin/Tcf-binding sites in FOP promoters). In a specific embodiment, at least one copy of a β-catenin/Tcf-binding site (also referred to as a Tcf/LEF-I binding site).

AT to GC changes in the FOP sequence abolish Tcf/LEF-1 binding and render the promoters non-responsive to β-catenin. To construct β-catenin/Tcf-responsive promoters, three copies of the Tcf/LEF-1 binding oligomers (TOP—SEQ ID NO: 52) were fused with minimal promoters from viral origins (TOP-CMV, TOP-TK), human cellular genes (TOP-hTERT, TOP-fos), or a combination of human and viral promoter elements (TOP-E2F-CMV). A corresponding control plasmid was constructed for each promoter by replacing the TOP oligomers with the mutant Tcf binding oligomers FOP (SEQ ID NO: 53; 5′-CCTTTGGCG-3′). TOP and FOP elements were generated by digestion of TOP-fos-LUC (TOPFLASH), FOP-fos-LUC (FOPFLASH), TOPTK-LUC, or FOPTK-LUC plasmids.

The activities of the promoters were measured with luciferase assays, and the results are indicated in FIG. 1B. Transfection experiments were normalized by the Dual Luciferase system (Promega). Control plasmid RL-TK (Promega) (0.2 μg) was used for normalization. Luciferase activities were measured 24 to 36 hours after transfection (means±s.d.) according to the manufacturer's instruction.

Except for TOP-hTERT, all β-catenin/Tcf-responsive promoters were selectively activated in colon cancer cell lines, given that their TOP/FOP ratios were much higher in the colon cancer cells (SW480 and DLD-1) than in liver-derived cells (Chang liver and SK-HEP-1). However, TOP-CMV exhibited much higher activity than any other β-catenin/Tcf-responsive promoters in the two colon cancer cell lines. Because of its high selectivity and activity in the colon cancer cell lines, TOP-CMV promoter was utilized as an exemplary construct in subsequent studies.

### Example 2

### Analysis of Multiple Adenoviral Vectors

In specific embodiments of the present invention, an adenoviral vector is the vector for the delivery system and a suicide gene, such as the HSV-TK gene, is the therapeutic gene. Four adenoviral vectors, AdCMV-luc, AdTOP-CMV-luc, AdCMV-TK, and AdTOP-CMV-TK were constructed. The adenoviral vectors were constructed by the AdEasy system (He et al., 1998b). The transcription termination sequences from the pGL3-Basic (Promega) and pcDNA3 plasmids (Invitrogen; Carlsbad, Calif.) were inserted into pShuttle plasmid in a tail-to-tail orientation to construct pShuttleGB. The promoters and reporter genes were then cloned into pShuttleGB vectors. Genomic adenoviral plasmids pAdCMV-luc, pAdTOP-CMV-luc, pAdCMV-TK, and pAdTOP-CMV-TK were generated by homologous recombination in E. coli strain BJ5183 from the pShuttleGB vectors. Adenovirus production and purification were performed by following standard procedures.

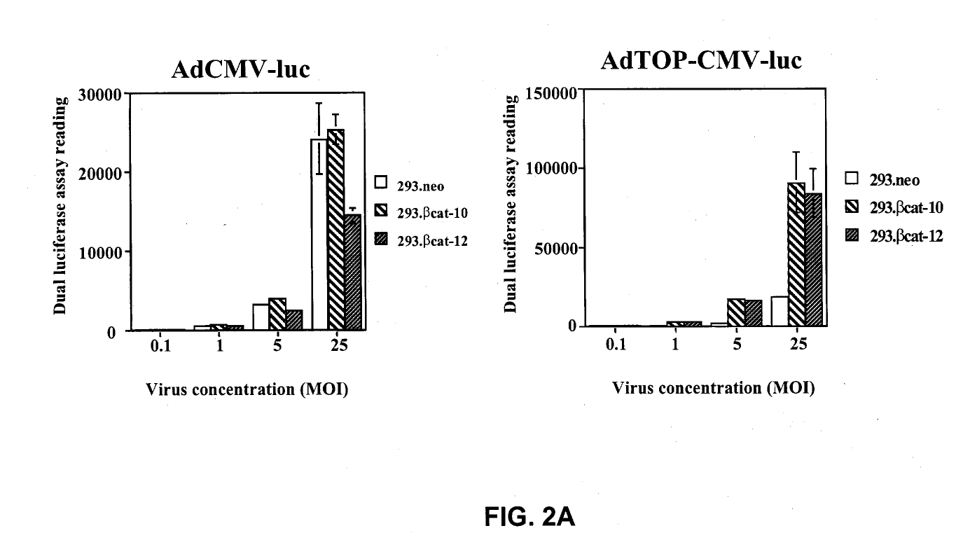

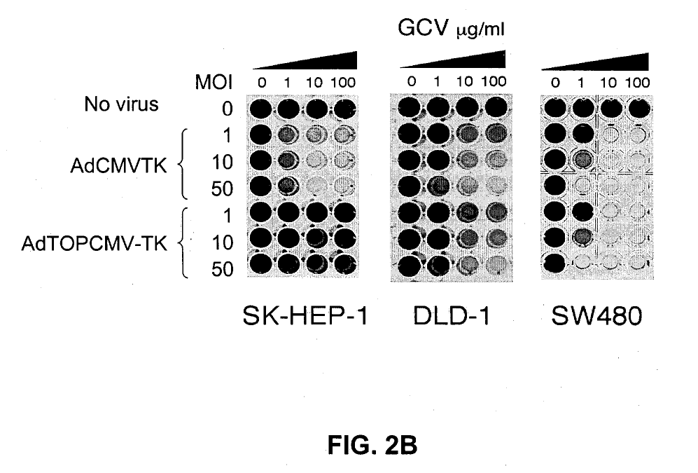

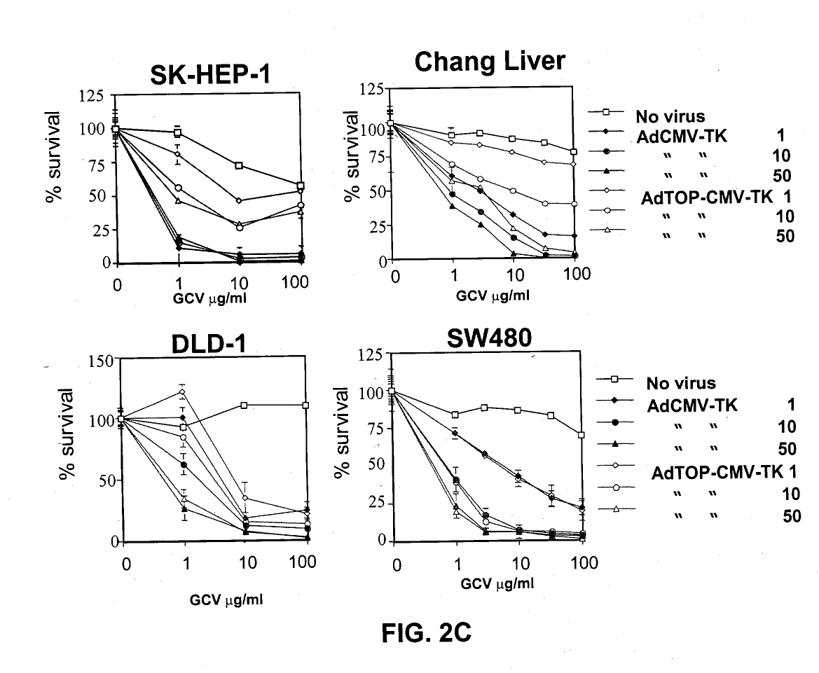

To test the selectivity of these adenoviral vectors, stable transfectants of HEK 293 cells bearing hyperactive β-catenin mutant (293.βcat-10 and 293.βcat-12) or selection marker only (293.neo) were infected with AdCMV-luc and AdTOP-CMV-luc. In FIG. 2A, HEK293 transfectant cell lines were infected with AdCMV-luc and AdTOP-CMV-luc viruses at various concentration (MOI, multiplicity of infection) and the luciferase activities were measured after 12 hours. 293. βcat-10 and 293.βcat-12 are two independent clones which expressed constitutively activated β-catenin mutant (S45Y), while 293.neo was vector transfectant. Luciferase activities were measured with Dual Luciferase system (Promega) 24 to 36 hours after infection (means±s.d.) according to the manufacturer's instruction.

As shown in FIG. 2A, the activity of AdTOP-CMV-luc was much stronger in β-catenin-hyperactive cells than in cells with basal β-catenin activity in luciferase assay. This result indicated that the adenoviral vector AdTOP-CMV could still selectively target β-catenin-hyperactive cells. The ability of AdCMV-TK and AdTOP-CMV-TK adenoviral vectors to kill cells with different β-catenin levels was compared by an in vitro assay. The four cell lines were infected with adenoviruses and treated with GCV 24 hours after viral infection. The cells were treated with GCV once daily for 7 days, and then cell viability was measured.

In FIG. 2B, Chang Liver (not shown in this picture), SK-HEP-1, DLD-1, and SW480 cells were infected with AdTOP-CMV-TK or AdCMV-TK viruses and treated with ganciclovir (GCV) once daily for 7 days. Twenty-four hours after adenoviral infection of cells in 96-well culture plates, culture medium was replaced by medium containing ganciclovir (GCV) (Roche, Basel, Switerland) once daily for 7 days. Cell viability was measured by the MTT (Sigma, St. Louis, Mo.) assay at the end of the 7 day treatment. The number of viable cells is proportional to the color intensities. The numbers on the right indicate the viral particles per cell for infection.

As shown in FIGS. 2B and 2C, cells with elevated β-catenin levels, such as SW480 and DLD-1, were killed efficiently by infection with either β-catenin/Tcf-responsive AdTOP-CMV-TK adenovirus or constitutively active AdCMV-TK adenovirus, plus GCV treatment. However, only AdCMV-TK, not AdTOP-CMV-TK, plus GCV treatment efficiently killed SK-HEP-1 and Chang liver cells, which were derived from liver origin. These results indicated that AdTOP-CMV-TK plus GCV treatment could be used in gene therapy to selectively kill colon cancer with little effect on liver.

### Example 3

### Ex Vivo Manipulations with Selective Adenovirus Vectors

To test the effectiveness of AdTOP-CMV-TK/GCV in suppressing tumor formation in animals, an ex vivo strategy was carried out. DLD-1 and SK-HEP-1 cells were infected with adenoviruses in vitro, harvested after 24 hours, and then inoculated subcutaneously into nude mice. The animals received intraperitoneal GCV treatment daily for 10 days, and the sizes of tumor were monitored twice per week.

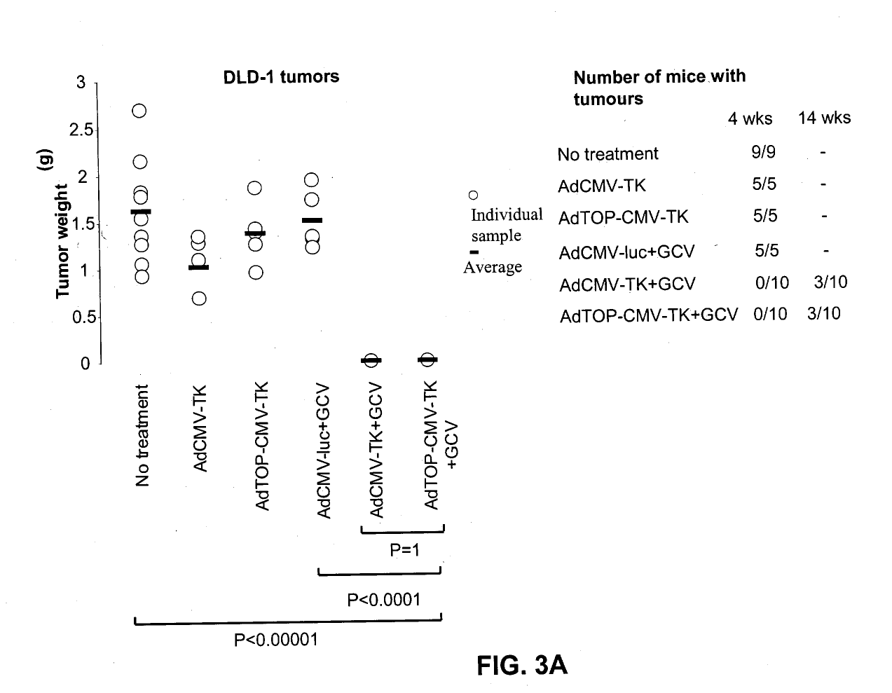

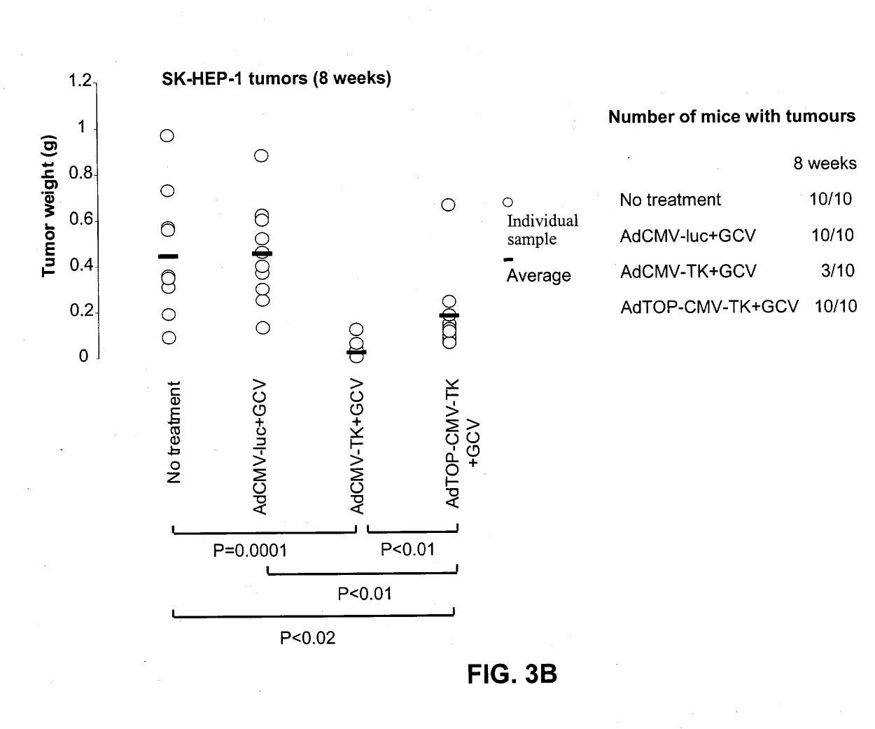

In FIG. 3A, human DLD-1 colon cancer cells were infected with 25 MOI of adenoviral vectors in serum free medium. Six to twelve hours after adenoviral infection, equal volumes of medium supplemented with 10% FBS were added to the infected cells, which were then incubated at 37° C. overnight. At 24 hours after adding the virus, the cells were trypsinized and inoculated subcutaneously into nude mice with 2×10⁶ DLD-1 cells per mouse. One day after inoculation of cancer cells, the mice in treatment groups received daily intraperitoneal injection of 2 mg of GCV in 0.5 ml 0.9% saline (approximately 100 mg/kg body weight) for 10 consecutive days. In two independent experiments, DLD-1 tumors in control groups reached 2 cm in diameter after 4 weeks and were killed in accordance with institutional animal policy. The tumors were dissected and their weights measured. Results from the two experiments were pooled and are shown in the same diagram.

As shown in FIG. 3A, both AdCMV-TK and AdTOP-CMV-TK viruses dramatically suppressed tumor growth with GCV treatment in DLD cells. However, the AdTOP-CMV-TK did not suppress SK-HEP-1 tumor growth as efficiently as AdCMV-TK even in the combination of GCV treatment (FIG. 3B). These results, indicating that AdTOP-CMV-TK indeed selectively kills colon cancer, were consistent with the hypothesis that suicide gene expression driven by the TOP-CMV promoter can effectively suppress the growth of tumor with APC mutations and that the tumor suppression effect is diminished in liver cells in which the β-catenin pathway is inactivated.

### Example 4

### Significance od the Present Invention

In a recent report (Chen and McCormick, 2001), a similar but nonidentical gene therapy strategy targeting colon cancer by a β-catenin/Tcf-responsive promoter was reported. In that study, the commonly used TOP-TK promoter was inserted into an adenoviral vector AdWt-Fd to drive the expression of the pro-apoptotic gene Fadd. Unlike their studies that have used HSV-TK core promoter in the constructs, the present invention combines the minimal CMV promoter and β-catenin/Tcf-responsive element as the TOP-CMV promoter in the construct. This simple manipulation significantly improved the activity of the β-catenin/Tcf-responsive promoter in the β-catenin-hyperactive cell lines, while still maintaining the specificity (FIG. 1B). Success of cancer gene therapy depends not only on the specificity, but also the expression level of the therapeutic gene. Thus, TOP-CMV promoter is preferable to TOP-TK promoter.

In addition to the improvement in promoter activity, a different therapeutic gene, thymidine kinase (TK), was utilized, including GCV treatment, to further enhance the expected efficiency of this gene therapy. In the cells expressing TK, GCV was converted into an active compound, which not only killed that cell but also neighboring ones by a bystander effect. As shown in FIG. 3, growth of colon cancers and hepatomas in the animal model was not influenced by infection of AdTOP-CMV-TK in the absence of GCV treatment. However, growth of the infected colon cancer cells, but not hepatoma cells, was significantly suppressed by GCV treatment.

Although mutations in APC gene are limited predominately to colon or rectal cancers, hyperactivity of β-catenin has been reported in other tumors like hepatocellular carcinomas, melanomas, pilomatricomas, breast cancer, etc. (de La Coste et al, 1998; Rubinfeld et al., 1997; Chan et al., 1999; Lin et al, 2000.). Since mutations in β-catenin also resulted in the activation of β-catenin/Tcf-responsive promoters, the gene therapy system described herein is also applicable to these tumors. In fact, the hepatocellular carcinoma cell line HepG2, in which β-catenin is mutated, was very sensitive to treatment with AdTOP-CMV-TK/GCV.

Thus, the present invention improves the activity of a β-catenin/Tcf-responsive promoter over known methods and shows that such promoter was selectively activated in colon cancer cells. Furthermore, the combination of AdTOP-CMV-TK adenovirus and GCV treatment selectively killed β-catenin-hyperactive colon cancer cells, but not liver cells, with low β-catenin activity in both tissue culture and an animal model. Thus, the present invention demonstrates that this gene therapy system has therapeutic potential for the treatment of cancers having an activated Wnt/β-catenin pathway, particularly metastatic colon cancer in the liver.

### Example 5

### Clinical Trials

This example is concerned with the development of human treatment protocols using the compositions described herein alone or in combination with other anti-cancer drugs. The vectors comprising the β-catenin/Tcf-responseive promoter comprising at least one Tcf/LEF-1 binding site operatively linked to a second promoter region and a nucleic acid sequence encoding am amino acid sequence of interest will be of use in the clinical treatment of various cancers. Such treatment will be particularly useful tools in anti-tumor therapy, for example, in treating patients with colon cancer, although it would also be useful for ovarian, breast, prostate, pancreatic, brain, and lung cancers. and so forth that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the composition in clinical trials.

Patients with advanced, metastatic colon, breast, epithelial, ovarian carcinoma, pancreatic, or other cancers chosen for clinical study will typically be at high risk for developing the cancer, will have been treated previously for the cancer which is presently in remission, or will have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the the inventive composition and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), Akt (phosphorylated and un-phosphorylated forms) and in the cells (antiangiogenic fusion proteins, peptides or polypeptides or nucleic acids encoding the same) may be assessed and recorded.

In the same procedure, the inventive composition may be administered alone or in combination with the other anti-cancer drug. The administration may be in the pleural/peritoneal cavity, directly into the tumor, or in a systemic manner. The starting dose may be 0.5 mg/kg body weight. Three patients may be treated at each dose level in the absence of grade>3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the antiangiogenic fusion protein, peptide, or polypeptide or a nucleic acid encoding the antiangiogenic fusion protein, peptide, or polypeptides, and the lot of anti-cancer drug exceed 5 EU/kg for any given patient.

The inventive composition and/or the other anti-cancer drug combination, may be administered over a short infusion time or at a steady rate of infusion over a 7 to 21 day period. The inventive composition infusion may be administered alone or in combination with the anti-cancer drug. The infusion given at any dose level wilt be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the inventive composition in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phopshorylated forms) and Akt (phosphorylated and non-phosphorylated forms), p185, etc.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no Mryelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, ki67 and Tunel assay to measure apoptosis, Akt) and in the cells (Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. An urinalysis may be performed every 4 weeks.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### Example 6

### Materials and Methods

The following descriptions provide exemplary materials and methods for practicing some embodiments of the present invention. A skilled artisan is well-equipped to adjust these methods and reagents to fit other intended embodiments within the scope of the invention.

Cell Culture

Chang liver cells were purchased from American Type Culture Collection (Manassas, Va.). DLD-1, SW480, and SK-HEP-1 cells were obtained from Dr. Li-Kuo Su (University of Texas M. D. Anderson Cancer Center, TX). The cell lines were maintained in a 1:1 mixture of Dulbecco's modified Eagle's medium (DMEM) and Ham's F12 extract supplemented with 10% fetal bovine serum (FBS) and penicillin/streptomycin/amphotericin B (PSA; Life Technology, Rockville, Md.)

Plasmid Construction

TOP-fox-LUC (TOPFLASH), FOP-fos-LUC (FOPFLASH), TOPTK-LUC, and FOPTK-LUC were generous gifts from Dr. Hans Clevers (University Hospital, Utrecht, Netherlands). A series of modified luciferase plasmids were generated by insertion of promoters in a promoterless luciferase plasmid. The promoterless luciferase plasmid was generated by insertion of Nhel/Xbal fragment of the firefly luciferase coding region from the pGL3-Basic plasmid (Promega, Madison, Wis.) into Nhel/Xbal-digested RL-null plasmid (Promega, Madison, Wis.).

Full-length human cytomegalovirus (CMV) promoter in the CMV-LUC plasmid was obtained from the RL-CMV plasmid (Promega). TOP-CMV-LUC and FOP-CMV-LUC plasmids were constructed by insertion of a Smal/EcoR1-digested minimal CMV promoter from the pTRE plasmid (Clontech) and the wild type or mutant TCF elements from TOP-TK-LUC and into the aforementioned promoterless luciferase plasmid. An Eag-1 fragment from the E2F-1 promoter plasmid (a generous gift from Dr. David Johnson, M.D. Anderson Cancer Center) containing E2F binding sites was inserted into TOP-CMV-LUC and FOP-CMV-LUC to obtain TOP-E2F-LUC and FOP-E2F-LUC, respectively. A 120-bp core promoter of human telomerase promoter (hTERT) was amplified by polymerase chain reaction (PCR) and ligated with TOP, FOP sequences to obtain TOP-hTERT-LUC and FOP-hTERT-LUC, respectively (the human telomerase promoter plasmid was obtained from Dr. Bacchetti, McMaster University, Canada).

MC1-TK expression plasmid containing HSV TK was kindly provided by Dr. Richard Behringer (M. D. Anderson Cancer Center, TX). HSV TK coding sequence was removed from this plasmid and used for construction of adenoviral vectors.

Construction of Plasmids and Recombinant Adenoviruses

The adenoviral vectors were constructed by the AdEasy system (He et al., 1998b). The AdEasy system was obtained from Dr. Tong-Chuan, Johns Hopkins Oncology Center, Baltimore, Md.). pShuttle plasmid was modified as follows: the transcription termination sequence was removed from the pGL3-Basic plasmid (Promega) by Notl and Bglll digestion and ligated to the same sites of the pShuttle vector. The bovine growth hormone gene transcription termination sequence was amplified by PCR from the pcDNA3 plasmid (Invitrogen; Carlsbad, Calif.) and ligated into the Bglll site of pShuttle plasmid. The pGL3-Basic and pcDNA3 transcription termination sequences were arranged in a tail-to-tail orientation and the modified pShuttle plasmid containing the tail-to-tail transcription termination sequence was renamed pShuttleGB. The expression cassette was removed from CMV-LUC by Bglll and BamHl digestion and ligated into Bglll site of pShuttleGB and this plasmid is named pShuttleCMVLUC to obtain pShuttleCMVLUC. The expression cassette from TOP-CMV LUC was cloned into pShuttleGB to obtain pShuttleTCFLUC. The HSV TK gene was removed from the MC1-TK plasmid and replaced the luciferase gene in pShuttleCMVLUC to obtain pShuttleCMVTK. The luciferase gene in the pShuttleTCFLUC plasmid was replaced by HSV TK gene to obtain pShuttle TCFTK. Genomic adenoviral plasmids pAdCMVLUC, pAdTCFLUC, pAdTCMVTK, and pAdTCFTK were generated by homologous recombination in E. coli strain BJ5183 from pShuttleCMVluc, pShuttleTCFluc, pShuttleCMVTK and pShuttleTCFTK, respectively. Adenovirus production and purification were performed by following standard procedures.

Transfections and Luciferase Assay

Transfection experiments were normalized by the Dual Luciferase system (Promega). Two control plasmids, RL-CMV and RL-TK (Promega), were used for normalization. In experiments normalized by CMV promoter Renilla luciferase plasmid (RL-CMV), 1.95 μg of tested plasmid was mixed with 0.05 μg of RL-CMV for transfection. In experiments normalized by HSVTK promoter Renilla luciferase (RL-TK), 1.8 μg of test plasmid was mixed with 0.2.μg of RL-TK for transfection. Luciferase assays were performed following the manufacturer's instruction (Dual Luciferase Reporter system; Promega).

In Vitro Cell Viability Assay

Cells were plated in 96-well tissue culture plates and infected with adenovirus. Twenty-four hours after infection, culture medium containing adenovirus was replaced by medium (DMEM/F12/10% FBS/PSA) containing GCV (Roche, Basel, Switerland). The cells were treated with GCV once daily for 7 days, and then cell viability was measured. Culture medium was removed from the wells and the cells were incubated with 100 μl of culture medium containing 1 mg/ml of 3,[4,5-dimethylthiazol-2-yl]-2,5-diphenyltetrazolium bromide (MTT; Sigma, St. Louis, Mo.) at 37° C. for 1-2 hours. Lysis buffer (20% SDS/50% N,N-dimethyl formamide; 100 μl) was added to each well and incubated at 37° C. overnight. Absorbance at 570 nm was measured and the well containing cells that received no adenovirus or GCV treatment was normalized as having 100% viability.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### Patents

U.S. Pat. No. 3,817,837

U.S. Pat. No. 3,850,752

U.S. Pat. No. 3,939,350

U.S. Pat. No. 3,996,345

U.S. Pat. No. 4,162,282

U.S. Pat. No. 4,275,149

U.S. Pat. No. 4,277,437

U.S. Pat. No. 4,310,505

U.S. Pat. No. 4,366,241

U.S. Pat. No. 4,683,202

U.S. Pat. No. 4,684,611

U.S. Pat. No. 4,728,575

U.S. Pat. No. 4,728,578

U.S. Pat. No. 4,737,323

U.S. Pat. No. 4,921,706

U.S. Pat. No. 4,952,500

U.S. Pat. No. 5,302,523

U.S. Pat. No. 5,322,783

U.S. Pat. No. 5,384,253

U.S. Pat. No. 5,464,765

U.S. Pat. No. 5,538,877

U.S. Pat. No. 5,538,880

U.S. Pat. No. 5,401,511

U.S. Pat. No. 5,550,318

U.S. Pat. No. 5,563,055

U.S. Pat. No. 5,580,859

U.S. Pat. No. 5,589,466

U.S. Pat. No. 5,591,616

U.S. Pat. No. 5,603,872

U.S. Pat. No. 5,610,042

U.S. Pat. No. 5,656,610

U.S. Pat. No. 5,672,344

U.S. Pat. No. 5,702,932

U.S. Pat. No. 5,736,524

U:S. Pat. No. 5,780,448

U.S. Pat. No. 5,786,214

U.S. Pat. No. 5,789,215

U.S. Pat. No. 5,849,718

U.S. Pat. No. 5,871,727

U.S. Pat. No. 5,879,703

U.S. Pat. No. 5,889,155

U.S. Pat. No. 5,925,565

U.S. Pat. No. 5,928,906

U.S. Pat. No. 5,935,819

U.S. Pat. No. 5,939,277

U.S. Pat. No. 5,945,100

U.S. Pat. No. 5,981,271

U.S. Pat. No. 5,994,136

U.S. Pat. No. 5,994,624

U.S. Pat. No. 5,998,600

U.S. Pat. No. 6,013,516

U.S. Pat. No. 6,046,173

U.S. Pat. No. 6,107,090

EPO 0273085

PCT Application WO 92/17598

PCT Application WO 94/09699

PCT Application WO 95/04738

### Publications

Almendro N, Bellon T, Rius C, Lastres P, et al. Cloning of the human platelet endothelial cell adhesion molecule-1 promoter and its tissue-specific expression. Structural and functional characterization. J Immunol 1996 Dec 15;157(12):5411-21.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Banerji et al., Cell, 27:299, 1981.

Banerji et al., Cell, 35:729, 1983.

Bangham, et al., “Diffusion of univalent Ions across the Lamellae of Swollen Phospholipids” J. Mol. Biol., 13:238-252, 1965.

Berkhout et al., Cell, 59:273, 1989.

Bienz M, Clevers H. (2000) Cell. 103, 311-20.

Blanar et al, EMBO J., 8:1139, 1989.

Bodine and Ley, EMBO J., 6:2997, 1987.

Boshart et al., Cell, 41:521, 1985.

Bosze et al, EMBO J., 5:1615, 1986.

Braddock et al., Cell, 58:269, 1989.

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Celander and Haseltine, J. Virology, 61:269, 1987.

Celander et al., J. Virology, 62:1314, 1988.

Chan, E. F., Gat, U., McNiff, J. M. & Fuchs, E. (1999). Nat Genet, 21, 410-3.

Chandler et al., Cell, 33:489, 1983.

Chang et al., Mol. Cell. Biol., 9:2153, 1989.

Chatte jee et al., Proc. Nat'l Acad. Sci. USA., 86:9114, 1989.

Chen, R. H., McConnick, F. (2001) Cancer Res., 61, 4445-9.

Choi et al., Cell, 53:519, 1988.

Cohen et al., “A Repetitive Sequence Element 3′ of the human c-Ha-ras1 Gene Has Enhancer Activity,” J. Cell. Physiol., 5:75, 1987.

Costa et al., Mol. Cell. Biol., 8:81, 1988.

Crawford, H. C., Fingleton, B. M., Rudolph-Owen, L. A., Goss, K. J., Rubinfeld, B., Polakis, P. & Matrisian, L. M. (1999). Oncogene, 18,2883-91.

Cripe et al., EMBO J., 6:3745, 1987.

Culotta and Hamer, Mol. Cell. Biol., 9:1376, 1989.

Dandolo et al., J. Virology, 47:55, 1983.

Deamer and P. Uster, “Liposome Preparation: Methods and Mechanisms,” in Liposomes (M. Ostro, ed.), Marcel Dekker, Inc., New York (1983), pp. 27-52.

Deamer and P. Uster, “Liposome Preparation: Methods and Mechanisms,” in Liposomes (M. Ostro, ed.), Marcel Dekker, Inc., New York (1983), pp. 27-52.

de La Coste, A., Romagnolo, B., Billuart, P., Renard, C. A., Buendia, M. A., Soubrane, O., Fabre, M., Chelly, J., Beldjord, C., Kahn, A. & Perret, C. (1998). Proc Natl Acad Sci USA, 95, 8847-51.

Deschamps et al., Science, 230:1174, 1985.

De Villiers et al., Nature, 312:242, 1984.

DRUG CARRIERS INBIOLOGYAND MEDICINE, G. Gregoriadis ed. (1979) pp. 287-341.

Edbrooke et al., Mol. Cell. Biol., 9: 1908, 1989.

Edlund et al., Science, 230:912, 1985.

Feng and Holland, Nature, 334:6178, 1988.

Firak and Subramanian, Mol. Cell. Biol., 6:3667, 1986.

Foecking and Hofstetter, “Powerful and/or Versatile Enhancer-Promoter Unit for [mammalian, plant, fungus, bacteria?] Expression Vectors,” Gene, 45:101, 1986.

Fraley and Fornari Kaplan, “Entrapment of a bacterial plasmid in phospholipid vesicles:potential for gene transfer,” Proc. Nat'l. Acad. Sci. USA 76:3348-3352, 1979.

Fujita et al., Cell, 49:357, 1987.

Gao, X. & Huang, L. (1991). Biochem Biophys Res Commun, 179, 280-5.

Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.

Gilles et al., Cell, 33:717, 1983.

Gloss et al., EMBO J., 6:3735, 1987.

Godbout et al., Mol. Cell. Biol., 8:1169, 1988.

Goodboum and Maniatis, Proc. Nat'l Acad. Sci. USA, 85:1447, 1988.

Goodbourn et al., Cell, 45:601, 1986.

Goss K H, Groden J. (2000). J. Clin Oncol. 18, 1967-79.

Greene et al., Immunology Today, 10:272, 1989.

Gregoriadis G. and Davis C. “Stability of liposomes in vivo and in vitro is promoted by their cholesterol content and the presence of blood cells,” Biochem Biophys Res Commun., 89(4):1287-1293, 1979.

Gregoriadis, DRUG CARRIERS IN BIOLOGY AND MEDICINE, G. Gregoriadis (ed.), 1979, pp. 287-341.

Grosschedl and Baltimore, Cell, 41:885, 1985.

Haslinger and Karin, Proc. Nat'l Acad. Sci. USA., 82:8572, 1985.

Hauber and Cullen, J. Virology, 62:673, 1988.

He, T. C., Sparks, A. B., Rago, C., Hermeking, H., Zawel, L., da Costa, L. T., Morin, P. J., Vogelstein, B. & Kinzler, K. W. (1998a). Science, 281, 1509-12.

He, T. C., Zhou, S., da Costa, L. T., Yu, J., Kinzler, K. W. & Vogelstein, B. (1998b). Proc Natl Acad Sci USA, 95, 2509-14.

Hen et al., Nature, 321:249, 1986.

Hensel et al., Lymphokine Res., 8:347, 1989.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Huang et al., Cell, 27:245, 1981.

Hug H, Costas M, Staeheli P, Aebi M, et al. Organization of the murine Mx gene and characterization of its interferon- and virus-inducible promoter. Mol Cell Biol 1988 Aug;8(8):3065-79.

Hwang et al, Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Johnson et al., Mol. Cell. Biol., 9:3393, 1989.

Kadesch and Berg, Mol. Cell. Biol., 6:2593, 1986.

Kaneda et al., “Increased expression of DNA cointroduced with nuclear protein in adult rat liver,” Science, 243:375-378, 1989.

Kaneda et al., “Introduction and expression of the human insulin gene in adult rat liver,” J. Biol Chem., 264(21):12126-12129, 1989.

Karin et al., Mol. Cell. Biol., 7:606, 1987.

Katinka et al., Cell, 20:393, 1980.

Katinka et al., Nature, 290:720, 1981.

Kato et al., “Expression of hepatitis B virus surface antigen in adult rat liver. Co-introduction of DNA and nuclear protein by a simplified liposome method,” J Biol Chem., 266(6):3361-3364, 1991.

Kawamoto et al., Mol. Cell. Biol., 8:267, 1988.

Kiledjian et al., Mol. Cell. Biol., 8:145, 1988.

Klamut et al., Mol. Cell. Biol., 10: 193, 1990.

Koch et al., Mol. Cell. Biol., 9:303, 1989.

Korinek et al., “Constitutive transcriptional activation by a β-catenin-Tcf complex in APC−/−colon carcinoma, Science, 275:1784-1787, 1997.

Kraus J, Woltje M, Schonwetter N, Hollt V. Alternative promoter usage and tissue specific expression of the mouse somatostatin receptor 2 gene. FEBS Lett 1998 May 29;428(3):165-70.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Landis, S. H., Murray, T., Bolden, S. & Wingo, P. A. (1998). Ca: a Cancer Journal for Clinicians, 48, 6-29.

Landis, S. H., Murray, T., Bolden, S. & Wingo, P. A. (1999). Ca: a Cancer Journal for Clinicians, 49, 8-31, 1.

Lareyre J J, Thomas T Z, Zheng W L, Kasper S, et al. A 5-kilobase pair promoter fragment of the murine epididymal retinoic acid-binding protein gene drives the tissue-specific, cell-specific, and androgen-regulated expression of a foreign gene in the epididymis of transgenic mice. J. Biol Chem 1999 Mar 19;274(12):8282-90.

Larsen et al., Proc. Nat l Acad. Sci. USA., 83:8283, 1986.

Laspia et al., Cell, 59:283, 1989.

Latimer et al., Mol. Cell. Biol., 10:760, 1990.

Lee et al., Mol. Endocrinol., 2: 404-411, 1988.

Lee et al., Nature, 294:228, 1981.

Lee S H, Wang W, Yajima S, Jose P A, et al. Tissue-specific promoter usage in the D1A dopamine receptor gene in brain and kidney. DNA Cell Biol 1997 Nov; 16(11):1267-75.

Levinson et al., Nature, 295:79, 1982.

Lin et al., Mol. Cell. Biol., 10:850, 1990.

Lin, S. Y., Xia, W., Wang, J. C., Kwong, K. Y., Spohn, B., Wen, Y., Pestell, R. G. & Hung, M. C. (2000). Proc Natl Acad Sci U S A, 97, 4262-6.

Liu W, Dong X, Mai M, Seelan R S, Taniguchi K, Krishnadath K K, Halling K C, Cunningham J M, Boardman L A, Qian C, Christensen E, Schmidt S S, Roche P C, Smith D I, Thibodeau S N., Mutations in AXIN2 cause colorectal cancer with defective mismatch repair by activating beta-catenin/TCF signalling. Nat Genet. 2000 Oct;26(2):146-7.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Majors and Varmus, Proc. Nat'l Acad. Sci. USA., 80:5866, 1983.

McNeall et al., Gene, 76:81, 1989.

Miksicek et al., Cell, 46:203, 1986.

Mordacq and Linzer, Genes and Dev., 3:760, 1989.

Moreau et al., Nucl. Acids Res., 9:6047, 1981.

Morin, P. J., Sparks, A. B., Korinek, V., Barker, N., Clevers, H., Vogelstein, B. & Kinzler, K. W. (1997). Science, 275, 1787-90.

Muesing et al., Cell, 48:691, 1987.

Ng et al., Nuc. Acids Res., 17:601, 1989.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al., “Liposomes as carriers for in vivo gene transfer and expression,” Methods Enzymol., 149:157-176, 1987.

Nomoto S, Tatematsu Y, Takahashi T, Osada H. Cloning and characterization of the alternative promoter regions of the human LIMK2 gene responsible for alternative transcripts with tissue-specific expression. Gene 1999 Aug 20;236(2):259-71.

Ondeket al., EMBO J., 6:1017, 1987.

Ornitz et al., Mol. Cell. Biol., 7:3466, 1987.

Palmiter et al., Nature, 300:611, 1982.

Pech et al., Mol. Cell. Biol., 9:396, 1989.

Perales et al., “Gene transfer in vivo: sustained expression and regulation of genes introduced into the liver by receptor-targeted uptake,” Proc. Natl. Acad. Sci. USA, 91:4086-4090, 1994.

Perez-Stable and Constantini, Mol. Cell. Biol., 10:1116, 1990.

Picard and Schaffter, Nature, 307:83, 1984.

Pinkert et al., Genes and Dev., 1:268, 1987.

Polakis, P. (2000). Genes Dev, 14, 1837-51.

Ponta et al., Proc. Nat'l Acad. Sci. USA., 82:1020, 1985.

Porton et al., Mol. Cell. Biol., 10: 1076, 1990.

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Resendez Jr. et al., Mol. Cell. Biol., 8:4579, 1988.

Ripe et al., Mol. Cell. Biol., 9:2224, 1989.

Rittling et al., Nucl. Acids Res., 17:1619, 1989.

Rosen et al., Cell, 41:813, 1988.

Rubinfeld, B., Robbins, P., El-Gamil, M., Albert, I., Porfiri, E. & Polakis, P. (1997). Science, 275, 1790-2.

Satake et al., “Biological Activities of Oligonucleotides Spanning the F9 Point Mutation Within the Enhancer Region of Polyoma Virus DNA,” J. Virology, 62:970, 1988.

Satoh S, Daigo Y, Furukawa Y, Kato T, Miwa N, Nishiwaki T, Kawasoe T, Ishiguro H, Fujita M, Tokino T, Sasaki Y, Imaoka S, Murata M, Shimano T, Yamaoka Y, Nakamura Y., AXINI mutations in hepatocellular carcinomas, and growth suppression in cancer cells by virus-mediated transfer of AXINI. Nat Genet. 2000 Mar;24(3):245-50.

Schaffner et al., J. Mol. Biol., 201:81, 1988.

Searle et al., Mol. Cell. Biol., 5:1480, 1985.

Sharp and Marciniak, Cell, 59:229, 1989.

Shaul and Ben-Levy, EMBO J., 6:1913, 1987.

Sherman et al., Mol. Cell. Biol., 9:50, 1989.

Shtutman, M., Zhurinsky,. J., Simcha, I., Albanese, C., D'Amico, M., Pestell, R. & Ben-Ze'ev, A. (1999). Proc Natl Acad Sci USA, 96, 5522-7.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Szoka and Papahadjopoulos, “Procedure for Preparation of Liposomes With Large Internal Aqueous Space . . . ”,Proc. Natl. Acad. Sci., 75:4194-4198, 1978.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Tetsu, O. & McCormick, F. (1999). Nature, 398, 422-6.

Thiesen et al., J. Virology, 62:614, 1988.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsumaki N, Kimura T, Tanaka K, Kimura J H, et al. Modular arrangement of cartilage- and neural tissue-specific cis- elements in the mouse alpha2(XI) collagen promoter. J. Biol Chem 1998 Sep 4;273(36):22861-4.

Tyndall et al., Nuc. Acids. Res., 9:6231, 1981.

Wagner et al., Science, 260:1510-1513, 1990.

Wong et al., “Appearance of beta-lactamase activity in animal cells upon liposome-mediated gene transfer,” Gene., 10(2):87-94, 1980.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Vannice and Levinson, J. Virology, 62:1305, 1988.

Vasseur et al., Proc. Nat'l Acad. Sci. USA., 77:1068, 1980.

Wang and Calame, Cell, 47:241, 1986.

Weber et al., Cell, 36:983, 1984.

Weinberger et al. Mol. Cell. Biol., 8:988, 1984.

Winoto and Baltimore, Cell, 59:649, 1989.

Wu H K, Squire J A, Song Q, Weksberg R. Promoter-dependent tissue-specific expressive nature of imprinting gene, insulin-like growth factor II, in human tissues. Biochem Biophys Res Commun 1997 Apr 7;233(1):221-6.

Yutzey et al. “An Internal Regulatory Element Controls Troponin I Gene Expression,” Mol. Cell. Biol., 9:1397, 1989.

Yutzey et al. Mol. Cell. Biol., 9:1397, 1989.

Zhao-Emonet J C, Boyer O, Cohen J L, Klatzmann D. Deletional and mutational analyses of the human CD4 gene promoter: characterization of a minimal tissue-specific promoter. Biochim Biophys Acta 1998 Nov 8;1442(2-3):109-19.

What is claimed is:

1. A viral vector, comprising:
a β-catenin/Tcf-responsive promoter construct comprising:
a first promoter region having at least one copy of a Tcf/LEF-1 binding site, operatively linked to;
a second promoter region; and
a nucleic acid sequence encoding an amino acid sequence of interest, wherein the first and second promoter regions are operatively linked to the target nucleic acid sequence.

2. The viral. vector of claim 1, further defined as an adenoviral vector.

3. The viral vector of claim 1, wherein the first promoter region comprises at least three copies of a Tcf/LEF-1 binding site.

4. The viral vector of claim 1, wherein the second promoter region is further defined as a minimal CMV promoter, TK promoter, fos promoter, or E2F promoter.

5. The viral vector of claim 1, wherein the second promoter region further comprises an E2F promoter.

6. The viral vector of claim 1, wherein the β-catenin/Tcf-responsive promoter comprises at least three copies of a Tcf/LEF-1 binding site and the second promoter region comprises a minimal CMV promoter.

7. The viral vector of claim 6, further defined as encoding a TOP-CMV promoter.

8. The viral vector of claim 1, wherein the nucleic acid sequence is further defined as a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof.

9. The viral vector of claim 8, wherein the nucleic acid sequence is further defined as encoding a suicide nucleic acid sequence.

10. The viral vector of claim 9, wherein the suicide nucleic acid sequence encodes thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, or E. coli Deo.

11. The viral vector of claim 8, wherein the nucleic acid sequence is further defined as encoding a cancer suppressor nucleic acid sequence, the cancer suppressor nucleic acid sequence further defined as encoding p53 or Rb.

12. The viral vector of claim 8, wherein the nucleic acid sequence is further defined as encoding a pro-apoptotic nucleic acid sequence, the pro-apoptotic nucleic acid sequence further defined as encoding p15, p16, or p21^{WAF-1}.

13. The viral vector of claim 8, wherein the nucleic acid sequence is further defined as encoding a cytokine nucleic acid sequence, the cytokine nucleic acid sequence further defined as encoding granulocyte macrophage colony stimulating factor, tumor necrosis factor α, interferon α, interferon γ, IL1, IL2, IL3, IL4, IL6, IL7, IL10, IL12, or IL15.

14. The viral vector of claim 1, further defined as being comprised in a pharmaceutical composition.

15. A nucleic acid segment comprising β-catenin/Tcf-responsive promoter construct comprising a first promoter region having a Tcf/LEF-1 binding site operatively linked to a second promoter, said second promoter being a minimal CMV promoter.

16. The nucleic acid segment of claim 15, wherein the first promoter region comprises at least three copies of a Tcf/LEF-1 binding site.

17. The nucleic acid segment of claim 15, further defined as encoding a TOP-CMV promoter.

18. The nucleic acid segment of claim 15, further defined as comprising a region encoding a polypeptide under the operative control of the β-catenin/Tcf-responsive promoter.

19. The nucleic acid segment of claim 18, wherein the polypeptide is further defined as a therapeutic polypeptide.

20. The nucleic acid segment of claim 18, wherein the region encoding a polypeptide is further defined as a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof.

21. The nucleic acid segment of claim 20, wherein the region encoding a polypeptide is further defined as a suicide nucleic acid sequence.

22. The nucleic acid segment of claim 21, wherein the suicide nucleic acid sequence encodes thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, or E. coli Deo.

23. The nucleic acid segment of claim 20, wherein the nucleic acid sequence is further defined as encoding a cancer suppressor nucleic acid sequence, the cancer suppressor nucleic acid sequence further defined as encoding p53 or Rb.

24. The nucleic acid segment of claim 20, wherein the nucleic acid sequence is further defined as encoding a pro-apoptotic nucleic acid sequence, the pro-apoptotic nucleic acid sequence further defined as encoding p15, p16, or p21^{WAF-1}.

25. The nucleic acid segment of claim 20, wherein the nucleic acid sequence is further defined as encoding a cytokine nucleic acid sequence, the cytokine nucleic acid sequence further defined as encoding granulocyte macrophage colony stimulating factor, tumor necrosis factor α, interferon α, interferon γ, IL1, IL2, IL3, TL4, IL6, IL7, IL10, IL12, or IL15.

26. The nucleic acid segment of claim 15, further defined as being comprised in a vector.

27. The nucleic acid segment of claim 26, further defined as comprised in a nonviral vector, a viral vector, or a combination thereof.

28. The nucleic acid segment of claim 27, wherein the viral vector is an adenoviral vector.

29. The nucleic acid segment of claim 27, wherein the viral vector is an adenoviral vector, a retroviral vector, or an adeno-associated viral vector.

30. The nucleic acid segment of claim 27, wherein the nonviral vector is a plasmid or a liposome.

31. The nucleic acid segment of claim 15, further defined as being comprised in a pharmaceutical composition.

32. A method of treating an individual with cancer, comprising administering to the individual a vector, said vector comprising:
a β-catenin/Tcf-responsive promoter construct comprising:
a first promoter region having at least one copy of a Tcf/LEF-1 binding site, operatively linked to;
a second promoter region; and
a nucleic acid sequence encoding a therapeutic polypeptide, wherein the first and second promoter regions are operatively linked to the nucleic acid sequence.

33. The method of claim 32, wherein the first promoter region comprises at least three copies of a Tcf/LEF-1 binding site.

34. The. method of claim 32, wherein the second promoter region comprises a minimal CMV promoter.

35. The method of claim 32, wherein the second promoter region comprises a minimal CMV promoter, TK promoter, fos promoter, or E2F promoter.

36. The method of claim 32, wherein the β-catenin/Tcf-responsive promoter comprises at least three copies of a Tcf/LEF-1 binding site and the second promoter region comprises a minimal CMV promoter.

37. The method of claim 32, wherein the second promoter region further comprises an E2F promoter.

38. The method of claim 32, wherein the nucleic acid sequence is further defined as a suicide nucleic acid sequence, a toxin nucleic acid sequence, a pro-apoptotic nucleic acid sequence, a cytokine nucleic acid sequence, an anti-angiogenic nucleic acid sequence, a cancer suppressor nucleic acid sequence, or a combination thereof.

39. The method of claim 32, wherein the therapeutic polypeptide is further defined as a suicide gene product.

40. The method of claim 38, wherein the nucleic acid sequence is further defined as encoding a suicide nucleic acid sequence, the suicide nucleic acid sequence further defined as encoding thymidine kinase, cytosine deaminase, p450 oxidoreductase, carboxypeptidase G2, β-glucuronidase, penicillin-V-amidase, penicillin-G-amidase, β-lactamase, nitroreductase, carboxypeptidase A, linamarase, E. coli gpt, or E. coli Deo.

41. The method of claim 38, wherein the nucleic acid sequence is further defined as encoding a cancer suppressor nucleic acid sequence, the cancer suppressor nucleic acid sequence further defined as encoding p53 or Rb.

42. The method of claim 38, wherein the nucleic acid sequence is further defined as encoding a pro-apoptotic nucleic acid sequence, the pro-apoptotic nucleic acid sequence further defined as encoding p15, p16, or p21^{WAF-1}.

43. The method of claim 38, wherein the nucleic acid sequence is further defined as encoding a cytokine nucleic acid sequence, the cytokine nucleic acid sequence further defined as encoding granulocyte macrophage colony stimulating factor, tumor necrosis factor α, interferon α, interferon γ, IL1, IL2, IL3, IL4, IL6, IL7, IL10, IL12, or IL15.

44. The method of claim 32, wherein the vector is comprised in a pharmaceutical composition.

45. The method of claim 32, wherein the vector is a viral vector.

46. The method of claim 45, wherein the viral vector is an adenoviral vector.

47. The method of claim 32, wherein the vector is a nonviral vector.

48. The method of claim 47, wherein the nonviral vector is a plasmid or a liposome.

49. The method of claim 32, further defined as comprising administering to the individual a prodrug.

50. The method of claim 49, wherein the prodrug is ganciclovir, acyclovir, FIAU [1-(2-deoxy-2-fluoro-β-D-arabinofuranosyl)-5-iodouracil], ifosfamide, 6-methoxypurine arabinoside, 5-fluorocytosine, doxorubicin, CB1954, nitrofurazone, N-(Cyanoacetyl)-L-phenylalanine, or N-(3-chloropropionyl)-L-phenylalanine.

51. The method of claim 32, wherein the cancer comprises a cell having a defective Wnt/β-catenin pathway.

52. The method of claim 32, wherein the cancer is colon cancer.

53. The method of claim 32, wherein the cancer is colon cancer that has metastasized to the liver.

54. The method of claim 32, further comprising administering to the individual chemotherapy, radiation, surgery, or gene therapy.

55. A method of treating colon cancer in an individual, comprising administering to the individual an adenoviral vector comprising:
a β-catenin/Tcf-responsive promoter construct comprising:
a first promoter region having three copies of a Tcf/LEF-1 binding site, operatively linked to;
a minimal CMV promoter; and
a nucleic acid sequence encoding thymidine kinase, wherein the first and second promoter regions are operatively linked to the nucleic acid sequence.

56. A method of screening for a modifier of β-catenin activity, comprising:
providing a β-catenin/Tcf-responsive promoter construct comprising:
a first promoter region having at least one copy of a Tcf/LEF-1 binding site, operatively linked to;
a second promoter; and
a reporter nucleic acid sequence, wherein the first and second promoter regions are operatively linked to the reporter nucleic acid sequence;
introducing to the vector a test compound; and
assaying for a change associated with the reporter nucleic acid sequence, wherein when said change occurs, said test compound is said modifier.

57. The method of claim 56, wherein said assaying step is defined as detecting transcription rate or level of said reporter nucleic acid sequence.

58. The method of claim 57, wherein when said transcription rate or level of said reporter nucleic acid sequence decreases, said test compound is an inhibitor of β-catenin activity.

59. The method of claim 56, wherein the reporter is green fluorescent protein, blue fluorescent protein, β-galactosidase, chloramphenicol acetyltransferase, or luciferase.

60. The method of claim 56, wherein the second promoter is minimal CMV promoter.

61. The method of claim 56, wherein the first promoter region comprises at least three copies of a Tcf/LEF-1 binding site.

62. The method of claim 56, wherein said test compound is a small molecule, a polypeptide, a polynucleotide, a sugar, a carbohydrate, a lipid, or a combination thereof.

63. The method of claim 56, wherein the method is further defined as occuring in a cell.

64. The method of claim 58, further comprising administering the inhibitor in a pharmaceutical composition to an individual having cancer related to a defective Wnt/β-catenin pathway.
